(19) United States
(12) Patent Application Publication
(10) Pub. No.: US 2026/0252878 A1
(43) Pub. Date: Aug. 27, 2026

(54) SYSTEM

(71) Applicant: SoftBank Group Corp., Tokyo (JP)

(72) Inventor: Yuko MIDORIKAWA, Tokyo (JP)

(73) Assignee: SoftBank Group Corp., Tokyo (JP)

(21) Appl. No.: 19/534,770

(22) Filed: Feb. 10, 2026

(30) Foreign Application Priority Data
Feb. 21, 2025 (JP) 2025-027015

(51) Int. Cl. G06N 3/08; G06N 20/00

(52) U.S. Cl. CPC G06N 3/08; G06N 20/00

(57) ABSTRACT

The system according to the embodiment comprises an imaging unit, a diagnosis unit, and a notification unit. The imaging unit captures images of the user's oral cavity using the PC's internal camera. The diagnosis unit uses AI to diagnose the images captured by the imaging unit. The notification unit sends the user the results of the oral check or a prompt to visit a medical institution based on the diagnosis result obtained by the diagnosis unit.

### CROSS-REFERENCE TO RELATED APPLICATION

The present application claims priority to and incorporates by reference the entire contents of Japanese Patent Application No. 2025-027015 filed in Japan on Feb. 21, 2025.

### BACKGROUND OF THE INVENTION

### 1. Field of the Invention

The technology of this disclosure relates to a system.

### 2. Description of the Related Art

Japanese Patent Application Laid-open No. 2022-180282 discloses a persona chatbot control method executed by at least one processor, comprising: receiving a user utterance, adding the user utterance to a prompt containing instructions related to the character of the chatbot, encoding the prompt, inputting the encoded prompt into a language model, and generating a chatbot utterance in response to the user utterance.

In conventional technology, busy businesspersons have difficulty undergoing regular dental checkups, resulting in insufficient management of oral health.

### SUMMARY OF THE INVENTION

The system according to the embodiment comprises an imaging unit, a diagnosis unit, and a notification unit. The imaging unit captures images of the user's oral cavity using the PC's internal camera. The diagnosis unit uses AI to diagnose the images captured by the imaging unit. The notification unit sends the user the results of the oral check or a prompt to visit a medical institution based on the diagnosis result obtained by the diagnosis unit.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a conceptual diagram showing an example configuration of a data processing system according to the first embodiment;

FIG. 2 is a conceptual diagram showing an example of main functions of a data processing device and a smart device according to the first embodiment;

FIG. 3 is a conceptual diagram showing an example configuration of a data processing system according to the second embodiment;

FIG. 4 is a conceptual diagram showing an example of main functions of a data processing device and smart glasses according to the second embodiment;

FIG. 5 is a conceptual diagram showing an example configuration of a data processing system according to the third embodiment;

FIG. 6 is a conceptual diagram showing an example of main functions of a data processing device and a headset-type terminal according to the third embodiment;

FIG. 7 is a conceptual diagram showing an example configuration of a data processing system according to the fourth embodiment;

FIG. 8 is a conceptual diagram showing an example of main functions of a data processing device and a robot according to the fourth embodiment;

FIG. 9 shows an emotion map where multiple emotions are mapped; and

FIG. 10 shows an emotion map where multiple emotions are mapped.

### DETAILED DESCRIPTION OF THE PREFERRED EMBODIMENTS

Hereinafter, an example of an embodiment of the system related to the technology disclosed herein will be described with reference to the attached drawings.

First, the terminology used in the following description will be explained.

In the following embodiments, a processor denoted by a reference numeral (hereinafter simply referred to as “processor”) may be a single computing device or a combination of multiple computing devices. The processor may be a single type of computing device or a combination of multiple types of computing devices. Examples of computing devices include a CPU (Central Processing Unit), GPU (Graphics Processing Unit), GPGPU (General-Purpose computing on Graphics Processing Units), APU (Accelerated Processing Unit), or TPU (Tensor Processing Unit), among others.

In the following embodiments, a RAM (Random Access Memory) denoted by a reference numeral is a memory where information is temporarily stored and used as a work memory by the processor.

In the following embodiments, a storage denoted by a reference numeral is one or more non-volatile storage devices for storing various programs and parameters. Examples of non-volatile storage devices include flash memory (SSD (Solid State Drive)), magnetic disks (e.g., hard disks), or magnetic tapes, among others.

In the following embodiments, a communication I/F (Interface) denoted by a reference numeral is an interface including a communication processor and an antenna, among others. The communication I/F manages communication between multiple computers. Examples of communication standards applicable to the communication I/F include wireless communication standards such as 5G (5th Generation Mobile Communication System), Wi-Fi (registered trademark), or Bluetooth (registered trademark), among others.

In the following embodiments, “A and/or B” means “at least one of A and B.” In other words, “A and/or B” means it may be only A, only B, or a combination of A and B. Moreover, when expressing three or more items connected by “and/or,” the same concept as “A and/or B” applies.

### First Embodiment

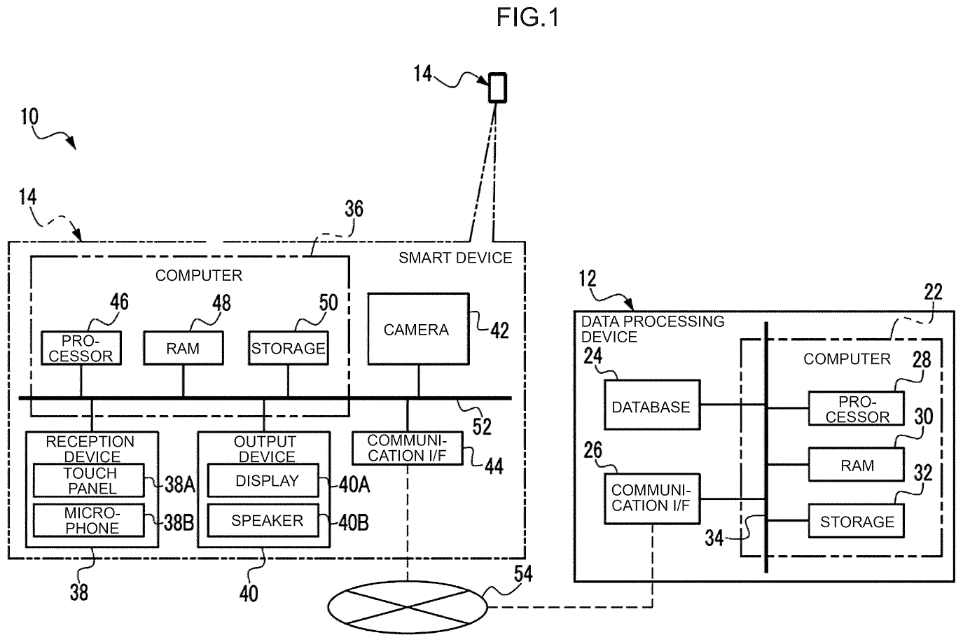

FIG. 1 shows an example configuration of a data processing system 10 according to the first embodiment.

As shown in FIG. 1, the data processing system 10 comprises a data processing device 12 and a smart device 14. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN (Wide Area Network) and/or a LAN (Local Area Network), among others.

The smart device 14 comprises a computer 36, a reception device 38, an output device 40, a camera 42, and a communication I/F 44. The computer 36 comprises a processor 46, RAM 48, and storage 50. The processor 46, RAM 48, and storage 50 are connected to a bus 52. The reception device 38, output device 40, and camera 42 are also connected to the bus 52.

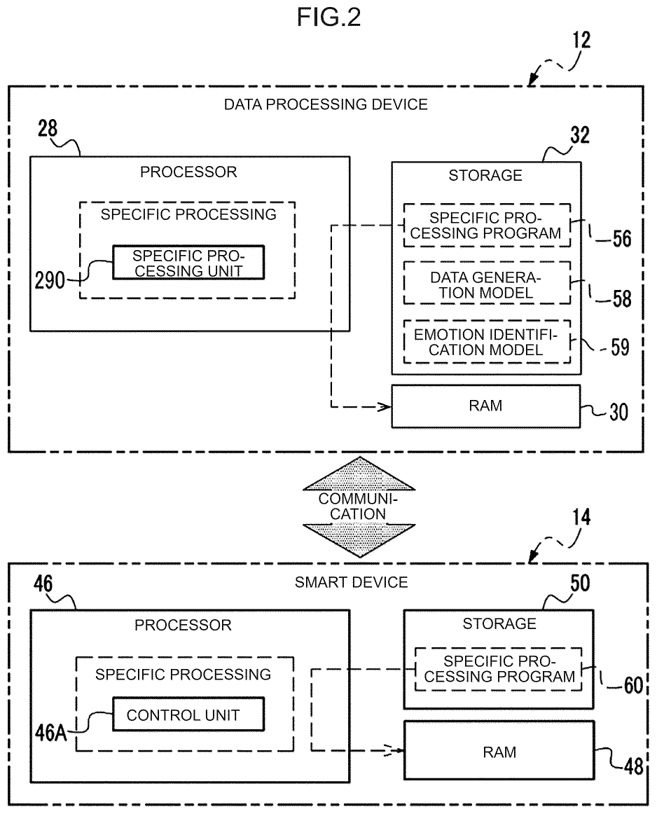

The reception device 38 comprises a touch panel 38A and a microphone 38B, among others, and accepts user input. The touch panel 38A accepts user input by detecting contact from an indicating object (e.g., a pen or finger). The microphone 38B accepts user input by detecting the user's voice. The control unit 46A sends data indicating user input accepted by the touch panel 38A and microphone 38B to the data processing device 12. The data processing device 12 has a specific processing unit 290 (see FIG. 2) that acquires data indicating user input.

The output device 40 comprises a display 40A and a speaker 40B, among others, and presents data to the user by outputting it in a perceptible form (e.g., audio and/or text). The display 40A displays visible information such as text and images according to instructions from the processor 46. The speaker 40B outputs audio according to instructions from the processor 46. The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors.

The communication I/F 44 is connected to the network 54. The communication I/F 44 and 26 manage the exchange of various information between the processor 46 and the processor 28 via the network 54.

FIG. 2 shows an example of the main functions of the data processing device 12 and the smart device 14.

As shown in FIG. 2, specific processing is performed in the data processing device 12 by the processor 28. The storage 32 stores a specific processing program 56. The specific processing program 56 is an example of a “program” related to the technology disclosed herein. The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart device 14, specific processing is performed by the processor 46. The storage 50 stores a specific processing program 60. The specific processing program 60 is used in conjunction with the specific processing program 56 by the data processing system 10. The processor 46 reads the specific processing program 60 from the storage 50 and executes it on the RAM 48. The specific processing is realized by the processor 46 operating as a control unit 46A according to the specific processing program 60 executed on the RAM 48. The smart device 14 may also have similar data generation models and emotion identification models as the data generation model 58 and emotion identification model 59, and perform the same processing as the specific processing unit 290 using these models.

Other devices besides the data processing device 12 may have the data generation model 58. For example, a server device (e.g., a generation server) may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.). Next, an example of processing by the data processing system 10 according to the first embodiment will be described.

### Example of the Embodiment

The health management system according to the embodiment of the present invention is a system that enables even busy businesspersons to receive regular dental checkups. This health management system allows a user to capture images of the oral cavity using a PC's internal camera, and the images are diagnosed by AI to perform oral checks and prompt visits to medical institutions. For example, when a user captures an image of the oral cavity with a PC's internal camera, the image is sent to AI, which diagnoses the condition of the oral cavity. Based on the diagnosis result, the user receives the results of the oral check or a prompt to visit a medical institution. In addition, the diagnosis result is linked with the company, enabling the company to support the health management of employees. With this system, even busy businesspersons can regularly check the health condition of their oral cavity and receive necessary treatment. For example, the user opens their mouth and captures an image facing the internal camera. This image is sent to AI. Next, the AI analyzes the sent image and diagnoses the condition of the oral cavity. For example, the AI detects signs of cavities or periodontal disease. Based on the diagnosis result, the user receives the results of the oral check or a prompt to visit a medical institution. For example, a message such as “Signs of cavities detected. Please visit a dental clinic.” is sent to the user. Furthermore, the diagnosis result is linked with the company, allowing the company to check the diagnosis results of employees and prompt visits to medical institutions if necessary. With this system, even busy businesspersons can regularly check the health condition of their oral cavity and receive necessary treatment. As a result, the health management system enables even busy businesspersons to regularly check the health condition of their oral cavity and receive necessary treatment. Specifically, this health management system acquires oral cavity image data captured by the user using a PC's internal camera (for example, an RGB 3-channel 224×224 pixel image tensor or higher-resolution image data) as input. During imaging, preprocessing such as brightness and contrast correction, noise removal, and automatic extraction of the oral region from the facial region can be performed. The AI diagnosis unit uses deep learning models such as convolutional neural networks (CNN) or multimodal Transformers to segment regions such as teeth, gums, and mucosa from the image and detect abnormal findings such as cavities, periodontal disease, and stomatitis. Examples of input to the AI include “Image captured by User A on Jun. 1, 2024” and “Enlarged image of User B's gum area.” The output of the AI is structured data such as abnormality detection labels (e.g., cavity present/absent, suspected periodontal disease, oral inflammation score 0.85), coordinate information of abnormal areas, and diagnosis confidence scores (0.0-1.0). For example, outputs such as “Suspected cavity in lower right molar No. 6 (score 0.92)” and “Signs of periodontal disease throughout the gums (score 0.78)” can be obtained. The diagnosis result is branched into actions such as “recommend visit to medical institution” or “follow-up observation” by threshold judgment (e.g., abnormality determined if score is 0.8 or higher) or rule-based post-processing. The notification unit automatically sends the diagnosis result to the user via email, SMS, or app notification, and sends the diagnosis data to the company's health management system via API integration or encrypted communication. On the company side, diagnosis histories for each employee are centrally managed on a health management dashboard, and instructions for visits or health guidance can be provided as needed. The AI model is trained in advance using a large number of oral cavity images and annotation data from specialists, employing loss functions such as cross-entropy loss and Dice loss, and is trained in a distributed manner on GPU clusters. Unlike conventional visual diagnosis by humans or manual health management, this system realizes integrated automatic extraction and analysis of high-dimensional image features, integration of multiple data sources, real-time diagnosis, notification, recording, and cooperation. As a result, technical effects such as improved diagnostic accuracy, significant reduction in processing speed, increased efficiency of data management, enhanced privacy protection, and reduced user burden are achieved. Specific application fields include employee health management in companies, remote health monitoring for teleworkers, pre-triage in dental clinics, group oral examinations in schools and elderly facilities, and health promotion programs by insurance companies.

The health management system according to the embodiment comprises an imaging unit, a diagnosis unit, and a notification unit. The imaging unit allows the user to capture images of the oral cavity using a PC's internal camera. To capture images of the oral cavity with a PC's internal camera, for example, the user opens their mouth and captures an image facing the internal camera. This image is sent to AI. The diagnosis unit analyzes the sent image using AI and diagnoses the condition of the oral cavity. For example, the AI detects signs of cavities or periodontal disease. The AI can use a text generation AI (e.g., LLM) to diagnose the condition of the oral cavity. In addition, the diagnosis unit can use a generative AI to diagnose the condition of the oral cavity. For example, the generative AI receives a prompt such as “Please diagnose the condition of the oral cavity in this image” and performs the diagnosis. The notification unit sends the results of the oral check or a prompt to visit a medical institution to the user based on the diagnosis result. For example, a message such as “Signs of cavities detected. Please visit a dental clinic.” is sent to the user. The notification unit can send notifications via email, SMS, app notification, and other methods. As a result, the health management system according to the embodiment enables even busy businesspersons to regularly check the health condition of their oral cavity and receive necessary treatment. Specifically, this health management system acquires oral cavity image data captured by the user using a PC's internal camera (for example, an RGB 3-channel 224×224 pixel image tensor or higher-resolution image data) as input. The imaging unit performs preprocessing such as brightness and contrast correction, noise removal, and automatic extraction of the oral region from the facial region during image acquisition. The diagnosis unit uses deep learning models such as convolutional neural networks (CNN) or multimodal Transformers to segment regions such as teeth, gums, and mucosa from the image and detect abnormal findings such as cavities, periodontal disease, and stomatitis. Examples of input to the AI include “Image captured by User A on Jun. 1, 2024” and “Enlarged image of User B's gum area.” The output of the AI is structured data such as abnormality detection labels (e.g., cavity present/absent, suspected periodontal disease, oral inflammation score 0.85), coordinate information of abnormal areas, and diagnosis confidence scores (0.0-1.0). For example, outputs such as “Suspected cavity in lower right molar No. 6 (score 0.92)” and “Signs of periodontal disease throughout the gums (score 0.78)” can be obtained. The diagnosis result is branched into actions such as “recommend visit to medical institution” or “follow-up observation” by threshold judgment (e.g., abnormality determined if score is 0.8 or higher) or rule-based post-processing. The notification unit automatically sends the diagnosis result to the user via email, SMS, or app notification, and sends the diagnosis data to the company's health management system via API integration or encrypted communication. The AI model is trained in advance using a large number of oral cavity images and annotation data from specialists, employing loss functions such as cross-entropy loss and Dice loss, and is trained in a distributed manner on GPU clusters. Unlike conventional visual diagnosis by humans or manual health management, this system realizes integrated automatic extraction and analysis of high-dimensional image features, integration of multiple data sources, real-time diagnosis, notification, recording, and cooperation. As a result, technical effects such as improved diagnostic accuracy, significant reduction in processing speed, increased efficiency of data management, enhanced privacy protection, and reduced user burden are achieved. Specific application fields include employee health management in companies, remote health monitoring for teleworkers, pre-triage in dental clinics, group oral examinations in schools and elderly facilities, and health promotion programs by insurance companies.

The health management system comprises a cooperation unit configured to cooperate with a company regarding the diagnosis result. The cooperation unit cooperates with the company regarding the diagnosis result. For example, the diagnosis result is sent to the company's health management system, enabling the company to support the health management of employees. The cooperation unit can use encryption technology to ensure data security when sending the diagnosis result to the company's health management system. For example, the diagnosis result is encrypted and sent to the company's health management system. By cooperating with the company regarding the diagnosis result, the company can support the health management of employees. Some or all of the above-described processing in the cooperation unit may be performed using AI or without using AI. For example, the cooperation unit may input the diagnosis result to an AI model and send it to the company's health management system. Specifically, the cooperation unit of this health management system receives structured diagnosis data output from the diagnosis unit (e.g., abnormality detection labels, abnormal area coordinates, diagnosis confidence scores in JSON or CSV format) and sends it to the API endpoint of the company's health management system using a secure communication channel such as HTTPS. Before sending, the cooperation unit encrypts the diagnosis data using public key cryptography such as AES or RSA to prevent eavesdropping or tampering during transmission. When using AI, the cooperation unit inputs the diagnosis result data, such as “Employee ID: 12345, Diagnosis Date: 2024 Jun. 1, Abnormality: Suspected cavity, Score: 0.92,” to the AI model, which automatically determines the optimal data mapping and transmission timing according to the database structure and employee management policy of the company's health management system. The AI model may be configured by combining a Transformer-based sequential data processing model and a rule-based data normalization module, and can perform preprocessing such as data item conversion, anonymization, aggregation, and splitting according to the content of the input data and the specifications of the destination system. Examples of input to the AI include “Diagnosis result data (JSON),” “API specification of the destination system,” and “Employee job attribute information.” The output of the AI includes control parameters such as “List of data items to be sent,” “Transmission timing (e.g., delayed transmission outside business hours),” “Selection of encryption method,” and the actual transmission data packet. For example, outputs such as “Encrypt diagnosis result with AES-256 and send at 18:00 on Jun. 1, 2024,” “Omit detailed data for employees in management positions” can be obtained. As post-processing, the cooperation unit sends data to the company's health management system's REST API or SOAP interface according to the AI output, and automates logging of transmission results and retransmission control in case of errors. Unlike conventional simple file transfer or manual data entry, this cooperation unit automates optimization of data content, transmission timing, and encryption method using AI, thereby achieving essential improvements in computer technology such as reduced communication load, enhanced security, improved data integrity, and increased efficiency of business processes. Technical effects include reduced risk of diagnosis data leakage, real-time health management cooperation, automation of individual responses for each employee, and increased flexibility in system cooperation. Specific application fields include employee health monitoring by corporate health management departments, remote health management for teleworkers, cooperation with industrial health systems, and health promotion programs by insurance companies.

The health management system comprises a consent unit configured to obtain the user's consent. The consent unit obtains the user's consent. For example, the user consents to linking the diagnosis result with the company. The consent unit can provide detailed explanations to deepen the user's understanding when obtaining consent. For example, detailed explanations are provided regarding the diagnosis content and data usage. In addition, the consent unit can record the user's consent history so that it can be referenced later. For example, the user's consent history is saved in a database and can be referenced as needed. By obtaining the user's consent, the diagnosis result can be used while protecting privacy. Some or all of the above-described processing in the consent unit may be performed using AI or without using AI. For example, the consent unit may input the user's consent to an AI model to obtain consent. Specifically, the consent unit of this health management system displays detailed explanation texts regarding the diagnosis content, purpose of data use, scope of third-party provision, retention period, etc. on the user interface, allowing the user to explicitly select options such as “Agree” or “Disagree.” When obtaining consent, the content of the explanation text can be customized according to the user's attributes (age, occupation, health status, etc.) and past consent history, and quizzes or confirmation dialogs can be inserted to check the level of understanding. When using AI, the consent unit inputs multidimensional data such as user operation logs, viewing time, questions, and past consent history (e.g., time-series event arrays, text input history, option vectors) to the AI model to estimate whether the user has sufficiently understood the content. The AI model may use a multimodal Transformer or BERT-based natural language understanding model to automatically summarize explanation texts, generate explanation texts according to user attributes, and optimize the consent acquisition process. Examples of input to the AI include “User A's consent screen viewing log,” “User B's questions and answer history,” and “Time required for past consent acquisition.” The output of the AI is structured data such as “Consent acquisition status (e.g., consented/not consented/needs re-explanation),” “Proposal for customizing explanation text,” and “Instruction to record consent history.” For example, outputs such as “User A has understood all items and consented,” “User B needs additional explanation” can be obtained. As post-processing, the consent unit records the consent history in a database (e.g., PostgreSQL, MongoDB) with a timestamp based on the AI output, and allows administrators or the user to reference the history as needed. Unlike conventional simple consent button presses or paper-based consent, this consent unit realizes optimization of explanation content, estimation of user understanding, and automatic management of consent history using AI, thereby improving computer technology such as enhanced privacy protection, individualized optimization of explanations, and increased efficiency of the consent acquisition process. Technical effects include improved accuracy of consent acquisition, prevention of trouble due to insufficient explanation, centralized management of consent history, and improved user experience. Specific application fields include consent for third-party provision of medical data, employee health management consent in companies, consent acquisition for insurance contracts, and consent management for remote medical services.

The health management system comprises a proposal unit configured to provide specific advice or treatment suggestions based on the diagnosis result. The proposal unit provides specific advice or treatment suggestions based on the diagnosis result. For example, a message such as “Signs of cavities detected. Please visit a dental clinic.” is sent to the user. The proposal unit can propose appropriate treatment methods to the user based on the diagnosis result. For example, a message such as “As a treatment method for cavities, fillings or crowns are suggested” is sent to the user. In addition, the proposal unit can provide specific advice based on the user's lifestyle and dietary habits. For example, a message such as “To prevent cavities, brush your teeth regularly and refrain from eating sweets” is sent to the user. By providing specific advice or treatment suggestions based on the diagnosis result, the user can receive appropriate treatment. Some or all of the above-described processing in the proposal unit may be performed using AI or without using AI. For example, the proposal unit may input the diagnosis result to an AI model to provide advice or treatment suggestions. Specifically, the proposal unit of this health management system integrates abnormality detection labels output from the diagnosis unit (e.g., suspected cavity, signs of periodontal disease, oral inflammation score), abnormal area coordinates, diagnosis confidence scores, and the user's lifestyle and dietary data (e.g., meal records, smoking history, frequency of tooth brushing), and inputs these to the AI model. The AI model may use algorithms such as a multimodal Transformer or gradient boosting decision tree, with diagnosis results and user attributes as input, to automatically generate treatment recommendations, self-care advice, and lifestyle improvement suggestions. Examples of input to the AI include “User A: suspected cavity in lower right molar No. 6 (score 0.92), smoking history, brushes teeth once a day,” “User B: signs of periodontal disease throughout the gums (score 0.78), low vegetable intake, frequent snacking.” The output of the AI includes structured data and natural language messages such as “Recommended treatment method (e.g., resin filling recommended for lower right molar No. 6, recommend visiting a dental clinic),” “Self-care advice (e.g., increase frequency of tooth brushing, recommend using floss),” “Lifestyle improvement suggestions (e.g., reduce frequency of snacking, recommend quitting smoking).” For example, outputs such as “Resin filling is recommended as a treatment for cavities. Please consider visiting a dental clinic,” “To prevent periodontal disease, brush your teeth after every meal and use floss” can be obtained. As post-processing, the proposal unit automatically adjusts the notification method (email, app notification, etc.) and the level of detail of the suggestions according to the user's preferences and visit history, and sends advice to the user in the optimal form. Unlike conventional uniform treatment suggestions or manual advice, this proposal unit realizes integrated analysis of diagnosis results and lifestyle data using AI, and provides individually optimized treatment and advice suggestions, thereby improving computer technology such as increased diagnostic accuracy, personalized treatment suggestions, and promotion of user behavioral change. Technical effects include improved accuracy of treatment suggestions, automation of self-care guidance, increased user satisfaction, and efficient use of medical resources. Specific application fields include employee health guidance in companies, remote medical services, health promotion programs by insurance companies, and health education in schools and elderly facilities.

The health management system comprises a recording unit configured to record the diagnosis result at regular intervals and compare it with past data. The recording unit records the diagnosis result at regular intervals and compares it with past data. For example, the diagnosis result is recorded monthly and compared with past data. The recording unit can also record the user's lifestyle and dietary data together with the diagnosis result. For example, the user's lifestyle data is recorded and changes in health condition are analyzed. In addition, the recording unit can save the user's health data in the cloud so that it can be accessed at any time. For example, the user's health data is saved in the cloud to prevent data loss. By recording the diagnosis result at regular intervals and comparing it with past data, changes in health condition can be grasped. Some or all of the above-described processing in the recording unit may be performed using AI or without using AI. For example, the recording unit may input the diagnosis result to an AI model and compare it with past data. Specifically, the recording unit of this health management system regularly records diagnosis result data output from the diagnosis unit or proposal unit (e.g., abnormality detection labels, abnormal area coordinates, diagnosis confidence scores) and the user's lifestyle and dietary data (e.g., meal records, exercise history, sleep time) in a time-series database (e.g., timestamped NoSQL database or time-series RDB). The recording unit automatically performs preprocessing such as data normalization, duplicate removal, and outlier detection during recording, and saves the data in encrypted form in cloud storage (e.g., AWS S3, Azure Blob) to prevent data loss or leakage. When using AI, the recording unit inputs past and new diagnosis data as time-series tensors (e.g., monthly abnormality score arrays, lifestyle vectors) to the AI model to automatically analyze trends in health condition and patterns of abnormality occurrence. The AI model may use time-series recurrent neural networks such as LSTM or GRU, autoregressive models, or anomaly detection algorithms (e.g., Isolation Forest) to compare with past data, analyze trends, and detect anomalies. Examples of input to the AI include “User A's diagnosis score array for the past 12 months,” “User B's lifestyle change vector,” and “History of abnormal area coordinates by diagnosis date.” The output of the AI includes structured data such as “Trend of changes in health condition (e.g., increasing abnormality score, effect of lifestyle improvement),” “Abnormality alert,” and “Summary of recorded content.” For example, outputs such as “Cavity risk has increased over the past 6 months,” “Periodontal disease score has decreased due to lifestyle improvement” can be obtained. As post-processing, the recording unit automatically generates and notifies health condition change reports to the user or administrator based on the AI output, and links to treatment suggestions or lifestyle improvement guidance as needed. Unlike conventional simple recording of diagnosis results or manual data comparison, this recording unit realizes time-series data analysis, anomaly detection, and automatic report generation using AI, thereby improving computer technology such as early detection of changes in health condition, increased efficiency of recording and analysis operations, and enhanced reliability of data management. Technical effects include visualization of changes in health condition, early detection of abnormalities, reduced risk of data loss, and improved user experience. Specific application fields include employee health monitoring in companies, follow-up observation in home medical care, health promotion programs by insurance companies, and health data analysis in research institutions.

The health management system comprises a sharing unit configured to share the diagnosis result with a professional dentist and obtain expert opinions. The sharing unit shares the diagnosis result with a professional dentist and obtains expert opinions. For example, the diagnosis result is sent to a professional dentist to request expert opinions. The sharing unit can also provide the user's past diagnosis data together when sharing the diagnosis result. For example, the user's past diagnosis data is provided together to obtain comprehensive opinions. In addition, the sharing unit can use encryption technology to ensure data security when sharing the diagnosis result. For example, the diagnosis result is encrypted and provided to the professional dentist. By sharing the diagnosis result with a professional dentist, expert opinions can be obtained. Some or all of the above-described processing in the sharing unit may be performed using AI or without using AI. For example, the sharing unit may input the diagnosis result to an AI model and provide it to the professional dentist. Specifically, the sharing unit of this health management system integrates diagnosis result data output from the diagnosis unit or recording unit (e.g., abnormality detection labels, abnormal area coordinates, diagnosis confidence scores) and the user's past diagnosis history, and sends these to a dedicated portal or API used by professional dentists. Before sending, the sharing unit encrypts the diagnosis data using encryption methods such as AES or RSA to prevent information leakage or tampering during transmission. When using AI, the sharing unit inputs diagnosis result data, past diagnosis history, and user attribute information (e.g., age, medical history) to the AI model, which automatically summarizes, highlights, and prioritizes the data to enable efficient diagnosis and advice by the professional dentist. The AI model may be configured by combining a natural language generation model for automatic summarization of diagnosis data and an image analysis model for automatic highlighting of abnormal areas. Examples of input to the AI include “User A's latest diagnosis result plus diagnosis history for the past year,” “List of coordinates of abnormal areas plus image data,” and “User's medical history and treatment history.” The output of the AI includes structured data and natural language text such as “Summary report for specialists,” “Highlighted images of abnormal areas,” and “Message requesting advice.” For example, outputs such as “Suspected cavity in lower right molar No. 6 (score 0.92), abnormality score increased over the past 6 months,” “Signs of periodontal disease, lifestyle improvement guidance provided” can be obtained. As post-processing, the sharing unit sends diagnosis data, summary reports, images, etc. to the professional dentist via a secure communication channel based on the AI output, and records and manages feedback or advice from the dentist. Unlike conventional simple transfer of diagnosis data or manual information organization, this sharing unit automates summarization, prioritization, and encrypted transmission of diagnosis data using AI, thereby improving computer technology such as increased efficiency of specialist diagnosis, enhanced data security, and increased efficiency of the opinion acquisition process. Technical effects include faster acquisition of expert opinions, improved diagnostic accuracy, reduced risk of data leakage, and reduced burden on doctors. Specific application fields include remote dental diagnosis services, cooperation with specialists in corporate health management, acquisition of second opinions for insurance company diagnoses, and sharing of diagnosis data between medical institutions.

The health management system comprises a reminder unit configured to send reminders to receive appropriate treatment. The reminder unit sends reminders to receive appropriate treatment. For example, a message such as “To receive treatment for cavities, please visit a dental clinic” is sent to the user. The reminder unit can select the optimal transmission timing by considering the user's schedule when sending reminders. For example, the user's schedule is referenced to select the optimal transmission timing. In addition, the reminder unit can select a transmission method according to the user's preferences when sending reminders. For example, if the user prefers email, the reminder is sent by email. By sending reminders to receive appropriate treatment, the user can receive treatment without forgetting. Some or all of the above-described processing in the reminder unit may be performed using AI or without using AI. For example, the reminder unit may input the reminder to an AI model to select the transmission timing. Specifically, the reminder unit of this health management system integrates diagnosis result data output from the diagnosis unit or proposal unit (e.g., abnormality detection labels, abnormal area coordinates, diagnosis confidence scores), the user's treatment history, and lifestyle data (e.g., visit history, scheduled treatment date, work calendar, private schedule), and inputs these to the AI model. The AI model may be configured by combining a Transformer-based sequential data processing model, a calendar optimization algorithm, and a notification channel selection module according to user attributes. Examples of input to the AI include “User A: suspected cavity in lower right molar No. 6 (score 0.92), scheduled treatment date Jul. 10, 2024, work calendar: weekdays 9-18,” “User B: signs of periodontal disease (score 0.78), no visit history, private schedule: family event on weekends.” The output of the AI includes structured data such as “Reminder transmission timing (e.g., Jul. 9, 2024, 20:00, after work),” “Transmission method (e.g., email, SMS, app notification),” and “Reminder content (e.g., treatment recommendation, candidate medical institution).” For example, outputs such as “Send ‘Don't forget your dental appointment tomorrow’ by email at 20:00 on Jul. 9, 2024,” “Since the weekend is busy, send a reminder by SMS on a weekday evening” can be obtained. As post-processing, the reminder unit automatically sends reminders to the user's device or application based on the AI output, and records and manages transmission history and read status. Unlike conventional fixed-time reminders or manual notifications, this reminder unit realizes integrated analysis of schedule, user attributes, and treatment history using AI, and automatic selection of optimal transmission timing and method, thereby improving computer technology such as increased reminder reception rate, prevention of missed treatments, reduced user burden, and increased efficiency of notification operations. Technical effects include increased treatment attendance rate, personalized reminders, improved user experience, and effective use of medical resources. Specific application fields include treatment reminders in employee health management in companies, support for visits by home medical care patients, programs to promote treatment compliance by insurance companies, and health management reminders in schools and elderly facilities.

The imaging unit can estimate the user's emotion and adjust the timing of imaging based on the estimated emotional state of the user. For example, if the user is relaxed, the imaging unit flexibly adjusts the timing of imaging and selects a timing that is easy for the user to capture images. In addition, if the user is feeling stressed, the imaging unit shortens the timing and completes imaging quickly. Furthermore, if the user is busy, the imaging unit can adjust the timing of imaging according to the user's schedule. By adjusting the timing of imaging based on the user's emotion, the user can capture images in a relaxed state. Emotion estimation is realized using, for example, an emotion engine or generative AI with emotion estimation functions. Generative AI may include text generation AI (e.g., LLM) or multimodal generative AI, but is not limited to these examples. Some or all of the above-described processing in the imaging unit may be performed using AI or without using AI. For example, the imaging unit may input the user's emotion data to generative AI and have the generative AI adjust the timing of imaging. Specifically, the imaging unit simultaneously acquires the user's facial expression image (e.g., RGB 3-channel facial image tensor 224×224 pixels), audio data (e.g., 5-second WAV format audio waveform), and biometric sensor data (e.g., heart rate, skin conductance values as numerical vectors), and inputs these to an emotion estimation AI model. The AI model may be configured by combining a multimodal Transformer, a ResNet-based facial expression recognition CNN, and an RNN for voice emotion classification. Examples of input include “User A's smiling image+normal heart rate+calm voice,” “User B's frowning image+high-stress heart rate+tense voice.” The output of the AI is structured data such as “Emotion label (e.g., relaxed, stressed, busy),” “Emotion score (e.g., relaxation level 0.85, stress level 0.72),” and “Recommended imaging timing (e.g., 5 minutes later, immediately, free time in user's calendar).” For example, outputs such as “User A is in a relaxed state (score 0.90), imaging timing can be flexibly adjusted,” “User B is in a stressed state (score 0.80), immediate imaging is recommended” can be obtained. As post-processing, the imaging unit links with the user's device calendar API or notification function based on the AI output and automatically sends imaging requests at the optimal timing. Unlike conventional fixed-time imaging or user-selected timing, this imaging unit combines multidimensional emotion estimation and scheduling optimization using AI, thereby achieving essential improvements in computer technology such as reduced user burden, increased cooperation rate for imaging, and stabilization of imaging data quality. Technical effects include improved user experience, increased efficiency of imaging data acquisition, prevention of data loss under stress, and overall system operation optimization. Specific application fields include stress-free health monitoring in employee health management in companies, reduced burden remote diagnosis for home medical care patients, increased cooperation rate in group examinations in schools and elderly facilities, and health promotion programs by insurance companies.

The imaging unit can emphasize and capture specific areas of the user's oral cavity during imaging. For example, if the user is suspected of having cavities, the imaging unit emphasizes and captures that area. In addition, if the user shows signs of periodontal disease, the imaging unit can emphasize and capture the condition of the gums. Furthermore, if the user complains of stomatitis, the imaging unit can emphasize and capture that area. By emphasizing and capturing specific areas, diagnostic accuracy is improved. Some or all of the above-described processing in the imaging unit may be performed using AI or without using AI. For example, the imaging unit may input instructions for emphasizing specific areas to an AI model and perform imaging. Specifically, the imaging unit acquires the entire oral cavity image of the user (e.g., RGB 3-channel high-resolution image tensor 512×512 pixels), and the AI model automatically detects and segments abnormal areas. The AI model may use deep learning models for image segmentation such as U-Net or Mask R-CNN to extract areas suspected of cavities, areas of gum inflammation, and areas of stomatitis at the pixel level. Examples of input to the AI include “User A's entire oral cavity image,” “User B's enlarged gum image.” The output of the AI is structured data such as “Coordinates of abnormal areas (e.g., bounding box for lower right molar No. 6),” “Instructions for emphasized imaging (e.g., zoom factor 2.0, change imaging angle by 30 degrees),” and “Imaging guide message (e.g., ‘Please capture a close-up of the lower right molar’).” For example, outputs such as “Recommend zoom imaging centered on suspected cavity area,” “Panoramic imaging instructions for entire gums” can be obtained. As post-processing, the imaging unit displays imaging guides on the user interface based on the AI output and performs automatic camera zoom/focus control and adjustment of imaging angle. Unlike conventional overall imaging or manual area specification, this imaging unit combines automatic detection of abnormal areas, generation of imaging guides, and optimization of camera control using AI, thereby improving computer technology such as increased diagnostic accuracy, reduced rate of re-imaging, and reduced user burden. Technical effects include high-precision imaging of abnormal areas, reduced diagnostic errors, increased efficiency of the imaging process, and improved user experience. Specific application fields include remote dental diagnosis services, abnormal area monitoring in corporate health management, pre-triage in dental clinics, and programs to improve diagnostic accuracy by insurance companies.

The imaging unit can measure the humidity and temperature of the user's oral cavity during imaging and utilize the measurements for diagnosis. For example, the imaging unit measures the humidity of the user's oral cavity and diagnoses dryness. In addition, the imaging unit measures the temperature of the user's oral cavity and diagnoses the presence or absence of inflammation. Furthermore, the imaging unit can comprehensively measure the humidity and temperature of the user's oral cavity and diagnose the health condition. By measuring the humidity and temperature of the oral cavity, diagnostic accuracy is improved. Some or all of the above-described processing in the imaging unit may be performed using AI or without using AI. For example, the imaging unit may input humidity and temperature data to an AI model and perform diagnosis. Specifically, the imaging unit acquires real-time temperature and humidity data (e.g., temperature 36.8° C., humidity 45%) from small sensors placed in the oral cavity (e.g., digital temperature sensor, capacitive humidity sensor), and inputs these together with image data (e.g., oral cavity image tensor) to the AI model. The AI model may use a multimodal Transformer or gradient boosting decision tree to perform integrated analysis of image features and sensor data. Examples of input to the AI include “User A's oral cavity image+temperature 36.5° C.+humidity 40%,” “User B's gum image+temperature 38.2° C.+humidity 30%.” The output of the AI is structured data such as “Dryness state label (e.g., normal/dry/severely dry),” “Inflammation risk score (e.g., 0.85),” and “Diagnosis comment (e.g., ‘Suspected oral dryness due to decreased humidity’).” For example, outputs such as “High inflammation risk due to increased temperature and decreased humidity,” “Within normal range” can be obtained. As post-processing, the imaging unit links data to the diagnosis unit or notification unit based on the AI output, and notifies the user of self-care advice or recommendation to visit a medical institution as needed. Unlike conventional diagnosis using only images or manual measurement of temperature and humidity, this imaging unit realizes integrated analysis of multimodal data and automatic diagnosis using AI, thereby improving computer technology such as increased diagnostic accuracy, earlier detection of abnormalities, and reduced user burden. Technical effects include early detection of oral abnormalities, increased reliability of diagnosis, automation of data acquisition, and improved user experience. Specific application fields include oral care monitoring in home medical care, support for diagnosis of inflammation and dryness in dental clinics, multidimensional health evaluation in corporate health management, and health promotion programs by insurance companies.

The imaging unit can estimate the user's emotion and adjust the frequency of imaging based on the estimated emotional state of the user. For example, if the user is relaxed, the imaging unit reduces the frequency of imaging to lessen the user's burden. In addition, if the user is feeling stressed, the imaging unit increases the frequency of imaging to collect more detailed data. Furthermore, if the user is busy, the imaging unit can adjust the frequency of imaging according to the user's schedule. By adjusting the frequency of imaging based on the user's emotion, the user's burden can be reduced. Emotion estimation is realized using, for example, an emotion engine or generative AI with emotion estimation functions. Generative AI may include text generation AI (e.g., LLM) or multimodal generative AI, but is not limited to these examples. Some or all of the above-described processing in the imaging unit may be performed using AI or without using AI. For example, the imaging unit may input the user's emotion data to generative AI and have the generative AI adjust the frequency of imaging. Specifically, the imaging unit regularly acquires the user's facial expression images, audio data, and biometric sensor data (e.g., heart rate, skin conductance values), and inputs these to an emotion estimation AI model. The AI model may use a multimodal Transformer or an LSTM-based time-series emotion estimation model to estimate the user's emotional state over time. Examples of input to the AI include “User A's facial expression images, audio, and heart rate time series for one week,” “User B's recent stress indicator trends.” The output of the AI is structured data such as “Emotion state label (e.g., relaxed, stressed, busy),” “Recommended imaging frequency (e.g., once a day, twice a week, once every two weeks),” and “Burden reduction score.” For example, outputs such as “User A has been in a relaxed state, so imaging frequency is reduced to once a week,” “User B's stress state is increasing, so imaging frequency is increased to twice a day” can be obtained. As post-processing, the imaging unit automatically generates an imaging schedule based on the AI output and sends reminders in cooperation with the user's device calendar or notification function. Unlike conventional fixed-frequency imaging or user-scheduled imaging, this imaging unit combines time-series analysis of emotional state and optimization of imaging frequency using AI, thereby improving computer technology such as minimization of user burden, increased efficiency of data collection, and prevention of data loss under stress. Technical effects include improved user experience, increased cooperation rate for imaging, stabilization of data quality, and increased efficiency of system operation. Specific application fields include burden-reducing monitoring in employee health management in companies, stress-considerate remote diagnosis for home medical care patients, increased cooperation rate in group examinations in schools and elderly facilities, and health promotion programs by insurance companies.

The imaging unit can capture the user's entire face during imaging and comprehensively determine the user's health condition based on complexion and facial expressions. For example, the imaging unit captures the user's complexion and determines signs of anemia or fatigue. In addition, the imaging unit captures the user's facial expressions and determines signs of stress or anxiety. Furthermore, the imaging unit can capture the user's entire face and comprehensively determine the health condition. By comprehensively determining the health condition based on complexion and facial expressions, more accurate diagnosis is possible. Some or all of the above-described processing in the imaging unit may be performed using AI or without using AI. For example, the imaging unit may input complexion and facial expression data to an AI model and perform health condition determination. Specifically, the imaging unit acquires the user's entire face image (e.g., RGB 3-channel facial image tensor 224×224 pixels), and automatically performs preprocessing such as face region detection, complexion extraction, and facial expression analysis. The AI model may use a ResNet or EfficientNet-based complexion analysis CNN, VGG-Face for facial expression recognition, or a multitask learning multimodal network to simultaneously output complexion features (e.g., redness, blueness, yellowness components), facial expression features (e.g., smile, frown, neutral expression), and health condition estimation scores (e.g., anemia risk 0.82, fatigue level 0.75, stress level 0.68). Examples of input to the AI include “User A's entire face image,” “User B's continuous facial expression images.” The output of the AI is structured data such as “Health condition label (e.g., suspected anemia, accumulated fatigue, high stress),” “Complexion score (e.g., redness 0.65, blueness 0.20),” and “Facial expression analysis result (e.g., smile rate 80%, frown rate 10%).” For example, outputs such as “Increased blueness in complexion indicates high anemia risk,” “Increased frown rate in facial expressions indicates signs of stress” can be obtained. As post-processing, the imaging unit links health condition data to the diagnosis unit or notification unit based on the AI output, and notifies the user of self-care advice or recommendation to visit a medical institution as needed. Unlike conventional visual diagnosis or manual evaluation of complexion and facial expressions, this imaging unit realizes automatic extraction of facial image features, health condition estimation, and multidimensional analysis using AI, thereby improving computer technology such as increased diagnostic accuracy, earlier detection of abnormalities, and reduced user burden. Technical effects include early detection of health abnormalities, increased reliability of diagnosis, automation of data acquisition, and improved user experience. Specific application fields include health monitoring in home medical care, employee health management in companies, health screening in schools and elderly facilities, and health promotion programs by insurance companies.

The imaging unit can record the user's voice during imaging and associate it with the health condition of the oral cavity. For example, the imaging unit records the user's voice and determines dryness of the oral cavity. In addition, the imaging unit records the user's voice and determines problems with tooth alignment or occlusion. Furthermore, the imaging unit can record the user's voice and comprehensively determine the health condition of the oral cavity. By recording the user's voice, the health condition of the oral cavity can be comprehensively determined. Some or all of the above-described processing in the imaging unit may be performed using AI or without using AI. For example, the imaging unit may input the user's voice data to an AI model and perform health condition determination. Specifically, the imaging unit records the user's speech audio data (e.g., 5-second WAV format audio waveform sampled at 16 kHz), and automatically performs acoustic feature extraction (e.g., Mel-frequency cepstral coefficients (MFCC), formant frequencies, spectral envelope) and speech recognition preprocessing. The AI model may use an RNN for voice emotion recognition, a CNN for acoustic anomaly detection, or a multimodal network for voice and image to output oral dryness state (e.g., increased friction sound during speech), abnormality in tooth alignment or occlusion (e.g., distorted pronunciation, difficulty pronouncing specific phonemes), and health condition estimation scores (e.g., dryness level 0.78, occlusion abnormality level 0.65). Examples of input to the AI include “User A's speech audio ‘a i u e o’,” “User B's continuous conversation audio.” The output of the AI is structured data such as “Dryness state label (e.g., normal/dry/severely dry),” “Occlusion abnormality label (e.g., normal/abnormal),” and “Health condition comment (e.g., ‘Suspected oral dryness based on speech sound’).” For example, outputs such as “High dryness risk due to increased friction sound,” “Suspected occlusion abnormality due to distorted pronunciation” can be obtained. As post-processing, the imaging unit links health condition data to the diagnosis unit or notification unit based on the AI output, and notifies the user of self-care advice or recommendation to visit a medical institution as needed. Unlike conventional manual voice evaluation or subjective health judgment, this imaging unit realizes automatic extraction of voice features, health condition estimation, and multidimensional analysis using AI, thereby improving computer technology such as increased diagnostic accuracy, earlier detection of abnormalities, and reduced user burden. Technical effects include early detection of health abnormalities based on voice, increased reliability of diagnosis, automation of data acquisition, and improved user experience. Specific application fields include voice health monitoring in home medical care, support for occlusion diagnosis in dental clinics, multidimensional health evaluation in corporate health management, and health promotion programs by insurance companies.

The diagnosis unit can estimate the user's emotion and adjust the manner of expressing the diagnosis result based on the estimated emotional state of the user. For example, if the user is relaxed, the diagnosis unit provides a detailed diagnosis result. In addition, if the user is feeling stressed, the diagnosis unit provides a concise diagnosis result. Furthermore, if the user is busy, the diagnosis unit provides a diagnosis result that focuses on the main points. By adjusting the manner of expressing the diagnosis result based on the user's emotion, the diagnosis unit can provide diagnosis results that are easy for the user to understand. Emotion estimation is realized using, for example, an emotion engine or generative AI with emotion estimation functions. Generative AI may include text generation AI (e.g., LLM) or multimodal generative AI, but is not limited to these examples. Some or all of the above-described processing in the diagnosis unit may be performed using AI or without using AI. For example, the diagnosis unit may input the user's emotion data to generative AI and have the generative AI adjust the manner of expressing the diagnosis result. Specifically, the diagnosis unit simultaneously acquires the user's facial expression image (e.g., RGB 3-channel 224×224 pixel facial image tensor), audio data (e.g., 5-second WAV format audio waveform), and biometric sensor data (e.g., heart rate, skin conductance values as numerical vectors), and inputs these to an emotion estimation AI model. The AI model may be configured by combining a multimodal Transformer, a ResNet-based facial expression recognition CNN, and an RNN for voice emotion classification. Examples of input to the AI include “User A's smiling image+normal heart rate+calm voice,” “User B's frowning image+high-stress heart rate+tense voice.” The output of the AI is structured data such as “Emotion label (e.g., relaxed, stressed, busy),” “Emotion score (e.g., relaxation level 0.85, stress level 0.72).” The diagnosis unit receives the output of the emotion estimation AI model and provides prompts such as “detailed diagnosis result,” “concise diagnosis result,” or “diagnosis result focusing on main points” to a diagnosis result generation AI (e.g., text generation LLM or summary generation model), and automatically switches the manner of expressing the diagnosis content. For example, for a user in a relaxed state, a detailed explanation such as “Suspected cavity in lower right molar No. 6 (score 0.92), resin filling recommended as treatment, self-care advice: brush teeth after every meal and use floss” is generated, while for a user in a stressed state, a concise explanation such as “Suspected cavity. Recommend visiting a dental clinic” is generated. Examples of AI output include “Detailed diagnosis report,” “Summary diagnosis comment,” and “Instruction for manner of expressing diagnosis result.” As post-processing, the diagnosis unit links the generated diagnosis result to the notification unit or recording unit and distributes it to the user device or management system in the optimal format. Unlike conventional uniform display of diagnosis results or manual adjustment of explanations, this diagnosis unit combines multidimensional emotion estimation and optimization of the manner of expressing diagnosis results using AI, thereby achieving essential improvements in computer technology such as automatic generation of diagnosis results that are easy for each user to understand, prevention of excessive or insufficient explanations, and improved user experience. Technical effects include personalized diagnosis results, optimization of explanations, increased user satisfaction, and increased efficiency of system operation. Specific application fields include stress-considerate diagnosis notifications in employee health management in companies, burden-reducing diagnosis reports for home medical care patients, optimization of explanations in group examinations in schools and elderly facilities, and health promotion programs by insurance companies.

The diagnosis unit can add a function to identify the types and quantities of bacteria in the oral cavity during diagnosis. For example, the diagnosis unit identifies the types of bacteria in the oral cavity and diagnoses the risk of cavities. In addition, the diagnosis unit identifies the quantity of bacteria in the oral cavity and diagnoses the risk of periodontal disease. Furthermore, the diagnosis unit can comprehensively identify the types and quantities of bacteria in the oral cavity and diagnose the health condition. By identifying the types and quantities of bacteria in the oral cavity, the risk of cavities and periodontal disease can be diagnosed. Some or all of the above-described processing in the diagnosis unit may be performed using AI or without using AI. For example, the diagnosis unit may input data on the types and quantities of bacteria to an AI model and perform diagnosis. Specifically, the diagnosis unit uses samples collected from the user's oral cavity (e.g., saliva, dental plaque, oral swab) to obtain bacterial DNA sequence data or 16S rRNA gene sequence data using a next-generation sequencer or real-time PCR device. The diagnosis unit vectorizes these sequence data and inputs them to an AI model (e.g., sequence classification model using convolutional neural networks, bacterial species identification model using random forest, clustering algorithms). Examples of input to the AI include “User A's 16S sequence data from saliva sample,” “User B's bacterial species count vector from dental plaque sample.” The output of the AI is structured data such as “List of bacterial species (e.g., Streptococcus mutans 35%, Porphyromonas gingivalis 10%),” “Total bacterial count (e.g., 1.2×10\hat{ }7 CFU/mL),” and “Risk score (e.g., cavity risk 0.85, periodontal disease risk 0.78).” For example, outputs such as “Increased Streptococcus mutans indicates high cavity risk,” “Detection of Porphyromonas gingivalis indicates high periodontal disease risk” can be obtained. The diagnosis unit performs threshold judgment or rule-based post-processing based on the AI output to determine actions such as “recommend visit to medical institution” or “strengthen self-care.” Unlike conventional culture methods or manual microscopic observation, this diagnosis unit realizes automatic analysis of high-dimensional sequence data, identification of bacterial species, and risk estimation using AI, thereby improving computer technology such as increased diagnostic accuracy, significant reduction in analysis speed, and increased efficiency of data management. Technical effects include high-precision diagnosis of cavity and periodontal disease risk, earlier detection of abnormalities, reduced user burden, and strengthened data cooperation with research institutions. Specific application fields include bacterial risk diagnosis in dental clinics, employee health management in companies, oral care monitoring in home medical care, health promotion programs by insurance companies, and oral microbiome analysis in research institutions.

The diagnosis unit can refer to the user's past diagnosis data during diagnosis to improve diagnostic accuracy. For example, the diagnosis unit refers to the user's past diagnosis data and compares it with the current diagnosis result. In addition, the diagnosis unit refers to the user's past diagnosis data to improve diagnostic accuracy. Furthermore, the diagnosis unit can comprehensively refer to the user's past diagnosis data and diagnose the health condition. By referring to past diagnosis data, diagnostic accuracy is improved. Some or all of the above-described processing in the diagnosis unit may be performed using AI or without using AI. For example, the diagnosis unit may input past diagnosis data to an AI model to improve diagnostic accuracy. Specifically, the diagnosis unit inputs diagnosis result data recorded for each user over time (e.g., abnormality detection labels, abnormal area coordinates, diagnosis confidence scores, lifestyle and dietary data) as time-series tensors or history vectors to the AI model. The AI model may use time-series recurrent neural networks such as LSTM or GRU, autoregressive models, or anomaly detection algorithms (e.g., Isolation Forest) to compare with past data, analyze trends, and detect anomalies. Examples of input to the AI include “User A's diagnosis score array for the past 12 months,” “User B's lifestyle change vector,” and “History of abnormal area coordinates by diagnosis date.” The output of the AI is structured data such as “Trend of changes in health condition (e.g., increasing abnormality score, effect of lifestyle improvement),” “Abnormality alert,” and “Instruction for correction of diagnosis content.” For example, outputs such as “Cavity risk has increased over the past 6 months,” “Periodontal disease score has decreased due to lifestyle improvement” can be obtained. The diagnosis unit corrects and emphasizes the current diagnosis result based on the AI output and clearly indicates trends and risk transitions to the user or medical personnel. As post-processing, the diagnosis unit links the corrected diagnosis result to the notification unit or recording unit and distributes it to the user device or management system in the optimal format. Unlike conventional single-time diagnosis or manual reference to history, this diagnosis unit realizes time-series data analysis, anomaly detection, and diagnosis correction using AI, thereby improving computer technology such as increased diagnostic accuracy, earlier detection of abnormalities, and reduced user burden. Technical effects include visualization of changes in health condition, increased reliability of diagnosis, increased efficiency of data management, and improved user experience. Specific application fields include employee health monitoring in companies, follow-up observation in home medical care, health promotion programs by insurance companies, and health data analysis in research institutions.

The diagnosis unit can estimate the user's emotion and adjust the level of detail of the diagnosis result based on the estimated emotional state of the user. For example, if the user is relaxed, the diagnosis unit provides a detailed diagnosis result. In addition, if the user is feeling stressed, the diagnosis unit provides a concise diagnosis result. Furthermore, if the user is busy, the diagnosis unit provides a diagnosis result that focuses on the main points. By adjusting the level of detail of the diagnosis result based on the user's emotion, the diagnosis unit can provide an appropriate diagnosis result for the user. Emotion estimation is realized using, for example, an emotion engine or generative AI with emotion estimation functions. Generative AI may include text generation AI (e.g., LLM) or multimodal generative AI, but is not limited to these examples. Some or all of the above-described processing in the diagnosis unit may be performed using AI or without using AI. For example, the diagnosis unit may input the user's emotion data to generative AI and have the generative AI adjust the level of detail of the diagnosis result. Specifically, the diagnosis unit simultaneously acquires the user's facial expression image (e.g., RGB 3-channel 224×224 pixel facial image tensor), audio data (e.g., 5-second WAV format audio waveform), and biometric sensor data (e.g., heart rate, skin conductance values as numerical vectors), and inputs these to an emotion estimation AI model. The AI model may be configured by combining a multimodal Transformer, a ResNet-based facial expression recognition CNN, and an RNN for voice emotion classification. Examples of input to the AI include “User A's smiling image+normal heart rate+calm voice,” “User B's frowning image+high-stress heart rate+tense voice.” The output of the AI is structured data such as “Emotion label (e.g., relaxed, stressed, busy),” “Emotion score (e.g., relaxation level 0.85, stress level 0.72).” The diagnosis unit receives the output of the emotion estimation AI model and provides prompts such as “detailed diagnosis result,” “concise diagnosis result,” or “diagnosis result focusing on main points” to a diagnosis result generation AI (e.g., text generation LLM or summary generation model), and automatically switches the level of detail of the diagnosis content. For example, for a user in a relaxed state, a detailed explanation such as “Suspected cavity in lower right molar No. 6 (score 0.92), resin filling recommended as treatment, self-care advice: brush teeth after every meal and use floss” is generated, while for a user in a stressed state, a concise explanation such as “Suspected cavity. Recommend visiting a dental clinic” is generated. Examples of AI output include “Detailed diagnosis report,” “Summary diagnosis comment,” and “Instruction for level of detail of diagnosis result.” As post-processing, the diagnosis unit links the generated diagnosis result to the notification unit or recording unit and distributes it to the user device or management system in the optimal format. Unlike conventional uniform display of diagnosis results or manual adjustment of explanations, this diagnosis unit combines multidimensional emotion estimation and optimization of the level of detail of diagnosis results using AI, thereby achieving essential improvements in computer technology such as automatic generation of appropriate diagnosis results for each user, prevention of excessive or insufficient explanations, and improved user experience. Technical effects include personalized diagnosis results, optimization of explanations, increased user satisfaction, and increased efficiency of system operation. Specific application fields include stress-considerate diagnosis notifications in employee health management in companies, burden-reducing diagnosis reports for home medical care patients, optimization of explanations in group examinations in schools and elderly facilities, and health promotion programs by insurance companies.

The diagnosis unit can correct the diagnosis result during diagnosis by considering the user's dietary habits and lifestyle. For example, the diagnosis unit considers the user's dietary habits and corrects the risk of cavities. In addition, the diagnosis unit considers the user's lifestyle and corrects the risk of periodontal disease. Furthermore, the diagnosis unit can comprehensively consider the user's dietary habits and lifestyle and correct the diagnosis result. By considering dietary habits and lifestyle, more accurate diagnosis results can be provided. Some or all of the above-described processing in the diagnosis unit may be performed using AI or without using AI. For example, the diagnosis unit may input dietary habits and lifestyle data to an AI model and perform correction of the diagnosis result. Specifically, the diagnosis unit vectorizes the user's recorded meal content (e.g., daily meal records, intake of carbohydrates, fats, vegetables), lifestyle data (e.g., frequency of tooth brushing, smoking history, drinking habits, sleep time, amount of exercise), and inputs these to the AI model (e.g., multimodal Transformer, gradient boosting decision tree, logistic regression). Examples of input to the AI include “User A: brushes teeth three times a day, frequent snacking, low vegetable intake,” “User B: smoking history, lack of sleep, no exercise habit.” The output of the AI is structured data such as “Diagnosis score after risk correction (e.g., cavity risk 0.85→0.92, periodontal disease risk 0.78→0.81),” “Comment on reason for correction (e.g., increased cavity risk due to increased frequency of snacking).” The diagnosis unit integrates the AI output with image diagnosis results and bacterial risk diagnosis results to correct and emphasize the final diagnosis result. As post-processing, the diagnosis unit links the corrected diagnosis result to the notification unit or proposal unit and provides the user with optimal treatment suggestions or self-care advice. Unlike conventional simple image diagnosis or manual consideration of lifestyle, this diagnosis unit realizes integration of multidimensional data, risk correction, and optimization of diagnosis using AI, thereby improving computer technology such as increased diagnostic accuracy, earlier detection of abnormalities, and reduced user burden. Technical effects include personalized diagnosis, improved accuracy of risk assessment, promotion of user behavioral change, and efficient use of medical resources. Specific application fields include employee health management in companies, lifestyle guidance in home medical care, health promotion programs by insurance companies, and health education in schools and elderly facilities.

The diagnosis unit can correct the diagnosis result by taking into account the user's genetic information during diagnosis. For example, the diagnosis unit considers the user's genetic information to correct the risk of dental caries. Additionally, the diagnosis unit can also correct the risk of periodontal disease by considering the user's genetic information. Furthermore, the diagnosis unit can comprehensively consider the user's genetic information to correct the diagnosis result. By considering genetic information, more accurate diagnosis results can be provided. Some or all of the above-described processes in the diagnosis unit may be performed using AI, or may be performed without using AI. For example, the diagnosis unit may input genetic information data into an AI model to execute correction of the diagnosis result. Specifically, the diagnosis unit vectorizes the user's genetic test data (e.g., SNP sequence data, whole genome sequencing data, specific gene polymorphism information, etc.) and inputs it into an AI model (e.g., sequence classification model using convolutional neural networks, risk estimation model using random forests, logistic regression, etc.). Examples of AI input include “User A: AMELX gene polymorphism, ENAM gene mutation” and “User B: IL-1β gene polymorphism, MMP-9 gene mutation.” The AI output consists of structured data such as “diagnosis score after genetic risk correction (e.g., caries risk 0.85→0.93, periodontal disease risk 0.78→0.88)” and “correction reason comments (e.g., increased risk of enamel hypoplasia due to AMELX polymorphism).” Based on the AI output, the diagnosis unit integrates the image diagnosis result and lifestyle risk diagnosis result to correct and emphasize the final diagnosis result. In subsequent processing, the diagnosis unit links the corrected diagnosis result to the notification unit and proposal unit to provide the user with optimal treatment suggestions and self-care advice. Unlike conventional simple image diagnosis or manual consideration of genetic information, the present diagnosis unit achieves improved diagnostic accuracy, earlier anomaly detection, and reduced user burden by implementing high-dimensional genetic data analysis, risk correction, and diagnosis optimization using AI, thereby improving computer technology. Technical effects include personalized diagnosis, improved accuracy of risk assessment, promotion of user behavioral change, and efficient use of medical resources. Specific application fields include employee health management in companies, genetic risk guidance in home healthcare, health promotion programs by insurance companies, and genetic epidemiology research in research institutions.

The notification unit can estimate the user's emotion and adjust the timing of notification based on the estimated emotion. For example, if the user is relaxed, the notification unit flexibly adjusts the timing of notification. If the user is feeling stressed, the notification unit can shorten the timing of notification. Furthermore, if the user is busy, the notification unit can adjust the timing of notification according to the user's schedule. By adjusting the timing of notification based on the user's emotion, the user can receive notifications at the optimal timing. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the notification unit may be performed using AI, or may be performed without using AI. For example, the notification unit may input the user's emotion data into generative AI to execute adjustment of notification timing. Specifically, the notification unit simultaneously acquires the user's facial expression images (e.g., RGB 3-channel 224×224 pixel facial image tensor), voice data (e.g., 5-second WAV format audio waveform), and biometric sensor data (e.g., heart rate, skin conductance values, etc.), and inputs these into an emotion estimation AI model. The notification unit uses an AI model combining a multimodal Transformer, ResNet-based facial expression recognition CNN, and RNN for voice emotion classification to estimate the user's emotional state with high accuracy. Examples of AI input include “User A's smiling image+normal heart rate+calm voice” and “User B's frowning image+high-stress heart rate+tense voice.” The AI output consists of structured data such as “emotion label (e.g., relaxed, stressed, busy),” “emotion score (e.g., relaxation level 0.85, stress level 0.72),” and “recommended notification timing (e.g., 5 minutes later, immediate, user's calendar free time).” For example, outputs such as “User A is in a relaxed state (score 0.90), notification timing can be flexibly adjusted” and “User B is in a stressed state (score 0.80), immediate notification is recommended” can be obtained. Based on the AI output, the notification unit automatically sends notification requests at the optimal timing by linking with the calendar API and notification functions of the user terminal. In subsequent processing, the notification unit records and manages notification history and read status, and dynamically adjusts notification content and frequency as needed. Unlike conventional fixed-time notifications or user-selected timing, the present notification unit combines multidimensional emotion estimation and scheduling optimization using AI to achieve essential improvements in computer technology, such as reducing user burden, increasing notification reception rates, and personalizing notification content. Technical effects include improved user experience, efficient acquisition of notification data, prevention of ignoring notifications under stress, and overall system operation optimization. Specific application fields include stress-free health notifications for employee health management in companies, burden-reducing remote notifications for home healthcare patients, improved cooperation in group health notifications at schools and elderly facilities, and health promotion programs by insurance companies.

The notification unit can select a notification method (such as email, SMS, or app notification) according to the user's preferences at the time of notification. For example, if the user prefers email, the notification unit sends notifications via email. If the user prefers SMS, the notification unit can also send notifications via SMS. Furthermore, if the user prefers app notifications, the notification unit can send notifications via app notification. By selecting the notification method according to the user's preferences, the user can receive notifications in the most accessible way. Some or all of the above-described processes in the notification unit may be performed using AI, or may be performed without using AI. For example, the notification unit may input notification method selection into an AI model to execute notification. Specifically, the notification unit collects multidimensional data such as the user's notification reception history, device type, app usage status, past notification read rates, and user attributes (age, occupation, work style, etc.), and inputs these into an AI model. The AI model, using methods such as gradient boosting decision trees, multimodal Transformers, or recurrent neural networks for user behavior prediction, automatically selects the optimal notification channel (email, SMS, app notification, etc.). Examples of AI input include “User A: notification read rate over the past 30 days (email 80%, SMS 95%, app notification 60%), device: smartphone” and “User B: high app usage frequency, SMS reception unavailable.” The AI output consists of structured data such as “recommended notification method (e.g., SMS, app notification, email),” “notification channel priority list,” and “notification content optimization instructions.” For example, outputs such as “SMS prioritized for User A, app notification also used” and “app notification only for User B” can be obtained. Based on the AI output, the notification unit automatically sends notifications via the user's device or system API, and records and analyzes the transmission results and read status. Unlike conventional uniform notification methods or user-selected channels, the present notification unit achieves improvements in computer technology by integrating analysis of user attributes, behavior history, and device information and optimizing notification channels using AI, resulting in increased notification reception rates, improved user satisfaction, and more efficient notification operations. Technical effects include personalized notifications, maximized reception rates, improved user experience, and enhanced system operation efficiency. Specific application fields include employee health management notifications in companies, multi-channel notifications for home healthcare patients, health promotion program notifications by insurance companies, and health notifications at schools and elderly facilities.

The notification unit can select the optimal notification timing by considering the user's schedule at the time of notification. For example, the notification unit refers to the user's schedule to select the optimal notification timing. The notification unit can also adjust the timing of notification according to the user's schedule. Furthermore, the notification unit can comprehensively consider the user's schedule to select the optimal notification timing. By selecting the notification timing based on the user's schedule, the user can receive notifications at the most accessible timing. Some or all of the above-described processes in the notification unit may be performed using AI, or may be performed without using AI. For example, the notification unit may input schedule data into an AI model to execute selection of notification timing. Specifically, the notification unit collects schedule data such as the user's work calendar, private appointments, meeting schedules, vacation information, and past notification read timings, and inputs these into an AI model. The AI model, using methods such as Transformer-based sequential data processing models, calendar optimization algorithms, or recurrent neural networks for user behavior prediction, automatically determines the optimal notification timing. Examples of AI input include “User A: weekday work 9-18, meeting schedule: 2024 Jul. 10 10:00-11:00, past notification read timing: 20:00” and “User B: weekend family events, weekday evening free.” The AI output consists of structured data such as “recommended notification timing (e.g., 2024 Jul. 9 20:00, after work),” “notification timing candidate list,” and “notification delay/immediate instructions.” For example, outputs such as “delay notification during meetings, send notification after work” and “weekend schedule is full, send notification on weekday evenings” can be obtained. Based on the AI output, the notification unit automatically sends notifications by linking with the user's device and calendar API, and records and manages transmission history and read status. Unlike conventional fixed-time notifications or manual scheduling, the present notification unit achieves improvements in computer technology by integrating analysis of schedules and user behavior history and optimizing notification timing using AI, resulting in increased notification reception rates, reduced user burden, and more efficient notification operations. Technical effects include personalized notification timing, maximized reception rates, improved user experience, and enhanced system operation efficiency. Specific application fields include employee health management notifications in companies, schedule-linked notifications for home healthcare patients, health promotion program notifications by insurance companies, and health notifications at schools and elderly facilities.

The notification unit can estimate the user's emotion and adjust the content of notification based on the estimated emotion. For example, if the user is relaxed, the notification unit provides detailed notification content. If the user is feeling stressed, the notification unit can provide concise notification content. Furthermore, if the user is busy, the notification unit can provide notification content that focuses on key points. By adjusting the content of notification based on the user's emotion, the user can receive notifications with optimal content. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the notification unit may be performed using AI, or may be performed without using AI. For example, the notification unit may input the user's emotion data into generative AI to execute adjustment of notification content. Specifically, the notification unit acquires the user's facial expression images, voice data, and biometric sensor data (e.g., heart rate, skin conductance values, etc.), and inputs these into an emotion estimation AI model. The AI model is constructed by combining a multimodal Transformer, ResNet-based facial expression recognition CNN, and RNN for voice emotion classification. Examples of AI input include “User A's smiling image+normal heart rate+calm voice” and “User B's frowning image+high-stress heart rate+tense voice.” The AI output consists of structured data such as “emotion label (e.g., relaxed, stressed, busy)” and “emotion score (e.g., relaxation level 0.85, stress level 0.72).” The notification unit receives the output of the emotion estimation AI model and provides prompts such as “detailed notification content,” “concise notification content,” or “notification content with only key points” to a notification content generation AI (e.g., text generation LLM or summary generation model), automatically switching the expression style of notification content. For example, for a user in a relaxed state, the system generates a detailed explanation such as “Suspected caries in lower right molar No. 6 (score 0.92), recommended treatment: resin filling, self-care advice: brush teeth and use floss after every meal,” while for a user in a stressed state, it generates a concise explanation such as “Suspected caries. Dental visit recommended.” Examples of AI output include “detailed notification report,” “summary notification comment,” and “notification content expression style instructions.” In subsequent processing, the notification unit delivers the generated notification content to the user terminal or management system in the optimal format, and records and manages notification history and read status. Unlike conventional uniform notification content or manual adjustment of explanations, the present notification unit combines multidimensional emotion estimation and notification content expression optimization using AI to automatically generate easily understandable notification content for each user, prevent excessive or insufficient explanations, and improve user experience, thereby achieving essential improvements in computer technology. Technical effects include personalized notification content, optimized explanations, improved user satisfaction, and enhanced system operation efficiency. Specific application fields include stress-considerate notifications for employee health management in companies, burden-reducing notifications for home healthcare patients, optimized explanations for group health notifications at schools and elderly facilities, and health promotion programs by insurance companies.

The notification unit can determine the priority of notification according to the user's health condition at the time of notification. For example, if the user's health condition is deteriorating, the notification unit increases the priority of notification. If the user's health condition is stable, the notification unit can lower the priority of notification. Furthermore, the notification unit can comprehensively consider the user's health condition to determine the priority of notification. By determining the priority of notification according to the user's health condition, important notifications can be received preferentially. Some or all of the above-described processes in the notification unit may be performed using AI, or may be performed without using AI. For example, the notification unit may input health condition data into an AI model to execute determination of notification priority. Specifically, the notification unit collects health condition data output from the diagnosis unit or recording unit (e.g., anomaly detection labels, coordinates of abnormal areas, diagnosis confidence scores, lifestyle and dietary data, etc.), and inputs these into an AI model. The AI model, using methods such as gradient boosting decision trees, logistic regression, or multimodal Transformers, scores the severity and urgency of the health condition and automatically determines the priority of notification. Examples of AI input include “User A: suspected caries (score 0.92), signs of periodontal disease (score 0.78), lifestyle: history of smoking” and “User B: stable health condition, low anomaly score.” The AI output consists of structured data such as “notification priority (e.g., high/medium/low),” “notification content urgency label,” and “notification transmission timing instructions.” For example, outputs such as “high-priority immediate notification for User A” and “low-priority periodic notification for User B” can be obtained. Based on the AI output, the notification unit automatically adjusts the order and content of notification transmission and delivers notifications to the user terminal or management system in the optimal format. In subsequent processing, the notification unit records and manages notification history and read status, and dynamically adjusts notification content and frequency as needed. Unlike conventional uniform notification priorities or manual urgency determination, the present notification unit achieves improvements in computer technology by integrating analysis of health condition data and optimizing notification priority using AI, resulting in prevention of missing important notifications, reduced user burden, and more efficient notification operations. Technical effects include personalized notification priority, immediate transmission of important notifications, improved user experience, and enhanced system operation efficiency. Specific application fields include employee health management notifications in companies, emergency notifications for home healthcare patients, health promotion program notifications by insurance companies, and health notifications at schools and elderly facilities.

The notification unit can add a function to send notifications to the user's family or medical personnel at the time of notification. For example, the notification unit sends notifications to the user's family to share health status. The notification unit can also send notifications to the user's medical personnel to share diagnosis results. Furthermore, the notification unit can send notifications to both the user's family and medical personnel to support comprehensive health management. By sending notifications to the user's family or medical personnel, comprehensive health management can be supported. Some or all of the above-described processes in the notification unit may be performed using AI, or may be performed without using AI. For example, the notification unit may input notification content into an AI model to execute transmission to family or medical personnel. Specifically, the notification unit collects diagnosis result data output from the diagnosis unit or recording unit (e.g., anomaly detection labels, coordinates of abnormal areas, diagnosis confidence scores, lifestyle and dietary data, etc.), and manages these by linking with family contact information and medical personnel contact information (e.g., email addresses, SMS numbers, medical institution API endpoints) registered in advance by the user. The notification unit uses an AI model (e.g., multimodal Transformer, gradient boosting decision tree, user attribute and relationship estimation module) to automatically optimize the granularity and expression style of notification content for each recipient, generating notification content such as “detailed diagnosis report,” “summary notification,” or “emergency alert.” Examples of AI input include “User A's diagnosis result+family contact list,” “User B's diagnosis result+primary physician API endpoint,” and “User C's diagnosis result+both family and medical personnel.” The AI output consists of structured data such as “notification content for family (e.g., ‘Signs of dental caries found in ○○'s oral cavity. Please support a medical visit’),” “diagnosis data packet for medical personnel (e.g., JSON including coordinates of abnormal areas, diagnosis confidence score, summary of past history),” and “transmission timing/channel instructions (e.g., SMS for family, immediate transmission via API for medical personnel).” For example, outputs such as “concise health status notification for family, detailed diagnosis data and treatment recommendations for medical personnel,” and “simultaneous transmission to both in case of high urgency” can be obtained. In subsequent processing, the notification unit automatically sends notifications to family or medical personnel devices or systems based on the AI output, and records and manages transmission history and read status. Unlike conventional notification only to the user or manual contact with family or medical personnel, the present notification unit achieves improvements in computer technology by optimizing content for each recipient, automating transmission, and managing history using AI, resulting in more efficient sharing of health information, rapid cooperation in emergencies, and strengthened support systems for family and medical personnel. Technical effects include multidimensional cooperation in health management, prevention of important information transmission failures, promotion of cooperation among users, family, and medical personnel, and enhanced system operation efficiency. Specific application fields include family cooperation in employee health management in companies, cooperation between family and primary physician for home healthcare patients, cooperation between guardians and medical institutions at schools and elderly facilities, and health promotion programs by insurance companies.

The cooperation unit can estimate the user's emotion and adjust the content of cooperation with the company based on the estimated emotional state of the user. For example, if the user is relaxed, the cooperation unit provides detailed cooperation content. If the user is feeling stressed, the cooperation unit can provide concise cooperation content. Furthermore, if the user is busy, the cooperation unit can provide cooperation content that focuses on key points. By adjusting the content of cooperation with the company based on the user's emotion, the user can cooperate with the company in the most optimal way. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the cooperation unit may be performed using AI, or may be performed without using AI. For example, the cooperation unit may input the user's emotion data into generative AI to execute adjustment of cooperation content. Specifically, the cooperation unit simultaneously acquires the user's facial expression images (e.g., RGB 3-channel 224×224 pixel facial image tensor), voice data (e.g., 5-second WAV format audio waveform), and biometric sensor data (e.g., heart rate, skin conductance values, etc.), and inputs these into an emotion estimation AI model. The AI model is constructed by combining, for example, a multimodal Transformer, ResNet-based facial expression recognition CNN, and RNN for voice emotion classification. Examples of AI input include “User A's smiling image+normal heart rate+calm voice” and “User B's frowning image+high-stress heart rate+tense voice.” The AI output consists of structured data such as “emotion label (e.g., relaxed, stressed, busy)” and “emotion score (e.g., relaxation level 0.85, stress level 0.72).” The cooperation unit receives the output of the emotion estimation AI model and provides prompts such as “detailed cooperation content,” “concise cooperation content,” or “cooperation content with only key points” to a cooperation content generation AI (e.g., text generation LLM or summary generation model), automatically switching the expression style of cooperation content. For example, for a user in a relaxed state, the system sends a detailed explanation such as “Suspected caries in lower right molar No. 6 (score 0.92), recommended treatment: resin filling, self-care advice: brush teeth and use floss after every meal” to the company's health management system, while for a user in a stressed state, it sends a concise explanation such as “Suspected caries. Dental visit recommended.” Examples of AI output include “detailed cooperation report,” “summary cooperation comment,” and “cooperation content expression style instructions.” In subsequent processing, the cooperation unit delivers the generated cooperation content to the company's health management system API or administrator terminal in the optimal format, and records and manages cooperation history and transmission status. Unlike conventional uniform cooperation content or manual adjustment of explanations, the present cooperation unit combines multidimensional emotion estimation and cooperation content expression optimization using AI to automatically generate easily understandable cooperation content for each user, prevent excessive or insufficient explanations, and improve business process efficiency, thereby achieving essential improvements in computer technology. Technical effects include personalized cooperation content, optimized explanations, improved user satisfaction, and enhanced system operation efficiency. Specific application fields include stress-considerate cooperation notifications for employee health management in companies, burden-reducing cooperation reports for home healthcare patients, optimized explanations for group health cooperation at schools and elderly facilities, and health promotion programs by insurance companies.

The cooperation unit can integrate with the company's health management system and share data at the time of cooperation. For example, the cooperation unit integrates with the company's health management system to share diagnosis data. The cooperation unit can also cooperate with the company's health management system to manage employee health status. Furthermore, the cooperation unit can integrate with the company's health management system to support comprehensive health management. By integrating with the company's health management system and sharing data, employee health management can be performed efficiently. Some or all of the above-described processes in the cooperation unit may be performed using AI, or may be performed without using AI. For example, the cooperation unit may input health management system data into an AI model to execute data sharing. Specifically, the cooperation unit receives structured diagnosis data output from the diagnosis unit or recording unit (e.g., anomaly detection labels, coordinates of abnormal areas, diagnosis confidence scores, lifestyle and dietary data, etc. in JSON or CSV format), and transmits it to the company's health management system API endpoint using secure communication channels such as HTTPS. Before transmission, the cooperation unit encrypts the diagnosis data using public key cryptography methods such as AES or RSA to prevent eavesdropping or tampering during transmission. When using AI, the cooperation unit inputs diagnosis result data, company health management system specifications, employee job attribute information, etc. into an AI model to automatically perform preprocessing such as data item conversion, anonymization, aggregation, or splitting, as well as optimization of transmission timing and destination. Examples of AI input include “diagnosis result data (JSON),” “destination system API specifications,” and “employee job attribute information.” The AI output consists of control parameters such as “list of data items to be transmitted,” “transmission timing (e.g., delayed transmission outside business hours),” “selection of encryption method,” and the actual transmission data packet. For example, outputs such as “encrypt diagnosis result with AES-256 and transmit at 18:00 on 2024 Jun. 1,” and “omit detailed data for employees with managerial positions” can be obtained. In subsequent processing, the cooperation unit transmits data to the company's health management system REST API or SOAP interface according to the AI output, and automates logging of transmission results and retransmission control in case of errors. Unlike conventional simple file transfer or manual data entry, the present cooperation unit automates optimization of data content, transmission timing, and encryption method using AI, thereby achieving essential improvements in computer technology such as reduced communication load, enhanced security, improved data integrity, and increased business process efficiency. Technical effects include reduced risk of diagnosis data leakage, real-time health management cooperation, automated individual response for each employee, and increased flexibility in system integration. Specific application fields include employee health monitoring by corporate health management departments, remote health management for teleworkers, integration with occupational health systems, and health promotion programs by insurance companies.

The cooperation unit can cooperate with the company's welfare program to support health management at the time of cooperation. For example, the cooperation unit cooperates with the company's welfare program to support health management. The cooperation unit can also integrate with the company's welfare program to manage employee health status. Furthermore, the cooperation unit can cooperate with the company's welfare program to support comprehensive health management. By cooperating with the company's welfare program to support health management, comprehensive management of employee health can be achieved. Some or all of the above-described processes in the cooperation unit may be performed using AI, or may be performed without using AI. For example, the cooperation unit may input welfare program data into an AI model to execute health management support. Specifically, the cooperation unit transmits health condition data output from the diagnosis unit or recording unit (e.g., anomaly detection labels, diagnosis confidence scores, lifestyle and dietary data, etc.) to the API or data integration interface of the welfare program management system. Before transmission, the cooperation unit automatically performs preprocessing such as data normalization, anonymization, and aggregation, and optimizes data items according to the specifications of the welfare program. When using AI, the cooperation unit inputs health condition data, welfare program usage history, employee attribute information, etc. into an AI model to automatically generate optimal program proposals and health management support content. Examples of AI input include “Employee A's diagnosis result+welfare program usage history” and “Employee B's health condition+program participation status.” The AI output consists of structured data such as “recommended program (e.g., dental checkup assistance, health promotion seminar invitation),” “cooperation data item list,” and “transmission timing/channel instructions.” For example, outputs such as “invite Employee A to dental checkup assistance program” and “recommend Employee B to participate in health promotion seminar” can be obtained. In subsequent processing, the cooperation unit transmits data to the welfare program management system based on the AI output, and automatically collects and records feedback data for usage status and effectiveness measurement. Unlike conventional manual program guidance or uniform health management support, the present cooperation unit achieves improvements in computer technology by integrating analysis of health condition, usage history, and employee attributes and optimizing support content using AI, resulting in individualized optimization of welfare programs, efficient health management, and improved employee satisfaction. Technical effects include increased utilization rate of welfare programs, maximized effectiveness of health promotion measures, automated data integration, and enhanced system operation efficiency. Specific application fields include promotion of corporate health management, health support for teleworkers, integration of health promotion programs by insurance companies, and integration with occupational health systems.

The cooperation unit can estimate the user's emotion and adjust the frequency of cooperation based on the estimated emotional state of the user. For example, if the user is relaxed, the cooperation unit reduces the frequency of cooperation. If the user is feeling stressed, the cooperation unit can increase the frequency of cooperation. Furthermore, if the user is busy, the cooperation unit can adjust the frequency of cooperation according to the user's schedule. By adjusting the frequency of cooperation based on the user's emotion, the user can cooperate with the company at the optimal frequency. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the cooperation unit may be performed using AI, or may be performed without using AI. For example, the cooperation unit may input the user's emotion data into generative AI to execute adjustment of cooperation frequency. Specifically, the cooperation unit periodically acquires the user's facial expression images, voice data, and biometric sensor data (e.g., heart rate, skin conductance values, etc.), and inputs these into an emotion estimation AI model. The AI model uses, for example, a multimodal Transformer or LSTM-based time-series emotion estimation model to estimate the user's emotional state over time. Examples of AI input include “User A's one week of facial expression images+voice+heart rate time series” and “User B's recent stress indicator trends.” The AI output consists of structured data such as “emotion state label (e.g., relaxed, stressed, busy),” “recommended cooperation frequency (e.g., once per day, twice per week, once every two weeks),” and “burden reduction score.” For example, outputs such as “reduce cooperation frequency to once per week for User A due to continued relaxed state” and “increase cooperation frequency to twice per day for User B due to increasing stress state” can be obtained. In subsequent processing, the cooperation unit automatically generates a cooperation schedule with the company's health management system based on the AI output, and executes data transmission at the optimal frequency in coordination with API integration and notification functions. Unlike conventional fixed-frequency cooperation or manual scheduling, the present cooperation unit combines time-series analysis of emotional state and optimization of cooperation frequency using AI to minimize user burden, improve data cooperation efficiency, and prevent delays in information transmission under stress, thereby achieving essential improvements in computer technology. Technical effects include personalized cooperation frequency, reduced operational burden, stabilized data quality, and enhanced system operation efficiency. Specific application fields include burden-reducing cooperation in employee health management in companies, stress-considerate data cooperation for home healthcare patients, improved cooperation in group health cooperation at schools and elderly facilities, and health promotion programs by insurance companies.

The cooperation unit can introduce encryption technology to protect the user's privacy at the time of cooperation. For example, the cooperation unit encrypts diagnosis data and sends it to the company. The cooperation unit can also use encryption technology when sending data to protect the user's privacy. Furthermore, the cooperation unit can encrypt the user's diagnosis data to prevent access by third parties. By introducing encryption technology to protect the user's privacy, data security can be ensured. Some or all of the above-described processes in the cooperation unit may be performed using AI, or may be performed without using AI. For example, the cooperation unit may input the use of encryption technology into an AI model to execute data encryption. Specifically, the cooperation unit receives health condition data output from the diagnosis unit or recording unit (e.g., anomaly detection labels, diagnosis confidence scores, lifestyle and dietary data, etc.), and applies public key cryptography methods such as AES, RSA, elliptic curve cryptography, or communication channel encryption technologies such as TLS/SSL before transmission. When using AI, the cooperation unit inputs the content of transmission data, destination system security requirements, user's privacy policy, etc. into an AI model to automatically determine the optimal encryption method, key management method, and whether to split or anonymize data. Examples of AI input include “diagnosis data content+destination system specifications+user privacy policy” and “transmission data size+communication channel safety.” The AI output consists of structured data such as “recommended encryption method (e.g., AES-256, RSA-2048),” “key management instructions,” and “data splitting/anonymization instructions.” For example, outputs such as “encrypt diagnosis data with AES-256 and manage keys with HSM” and “split highly confidential data and send via separate channels” can be obtained. In subsequent processing, the cooperation unit executes encryption processing based on the AI output, sends data to the company's health management system via a secure communication channel, and records and manages transmission history and decryption status. Unlike conventional simple password protection or manual encryption, the present cooperation unit automates optimization of encryption method according to data content, destination, and policy using AI, thereby achieving essential improvements in computer technology such as reduced risk of data leakage, enhanced security, and improved operational efficiency. Technical effects include strengthened privacy protection, ensured data security, improved reliability of system integration, and automated legal compliance. Specific application fields include corporate health management data integration, secure data transmission in home healthcare, health promotion programs by insurance companies, and confidential data integration with research institutions.

The cooperation unit can anonymize the user's health data and provide it to the company at the time of cooperation. For example, the cooperation unit anonymizes the user's health data and provides it to the company. The cooperation unit can also anonymize health data and send it to protect the user's privacy. Furthermore, the cooperation unit can anonymize the user's health data and integrate it into the company's health management system. By anonymizing the user's health data and providing it to the company, health management can be performed while protecting privacy. Some or all of the above-described processes in the cooperation unit may be performed using AI, or may be performed without using AI. For example, the cooperation unit may input the use of anonymization technology into an AI model to execute data anonymization. Specifically, the cooperation unit receives health data output from the diagnosis unit or recording unit (e.g., anomaly detection labels, diagnosis confidence scores, lifestyle and dietary data, vital signs, etc. as structured data), and inputs it into an anonymization processing module. The anonymization processing module is constructed by combining algorithms such as k-anonymity, differential privacy, attribute masking, and ID tokenization. When using AI, the cooperation unit inputs health data content, destination system privacy policy, user attributes (age, occupation, position, etc.), and past anonymization history into an AI model (e.g., gradient boosting decision tree, multimodal Transformer, rule-based anonymization decision module) to automatically determine which data items should be anonymized and to what extent. Examples of AI input include “User A's diagnosis data+occupation: manager+destination: health management department” and “User B's lifestyle data+destination: welfare department.” The AI output consists of structured data such as “list of items to be anonymized (e.g., mask name, date of birth, employee number; range diagnosis score),” “anonymization method (e.g., k-anonymity with k=5, differential privacy ε=0.5),” and “transmission permission decision.” For example, outputs such as “delete name and employee number, range diagnosis score in increments of 0.1,” and “send only aggregated values for lifestyle data” can be obtained. In subsequent processing, the cooperation unit executes anonymization processing based on the AI output, and sends data to the company's health management system API or database via a secure communication channel. After transmission, anonymization logs and transmission history are recorded, and anonymization level audits or re-anonymization are automated as needed. Unlike conventional simple ID deletion or manual anonymization, the present cooperation unit automates optimization of anonymization method according to data content, destination, and policy using AI, thereby achieving essential improvements in computer technology such as strengthened privacy protection, promotion of data utilization, and improved operational efficiency. Technical effects include reduced risk of health data leakage, automated legal compliance, improved reliability of system integration, and increased user trust. Specific application fields include corporate health management data integration, secure data sharing in home healthcare, health promotion programs by insurance companies, and anonymized data integration with research institutions.

The consent unit can estimate the user's emotion and adjust the method of obtaining consent based on the estimated emotional state of the user. For example, if the user is relaxed, the consent unit provides a detailed explanation and obtains consent. If the user is feeling stressed, the consent unit can provide a concise explanation and obtain consent quickly. Furthermore, if the user is busy, the consent unit can provide an explanation focusing on key points and obtain consent. By adjusting the method of obtaining consent based on the user's emotion, consent can be obtained in the most optimal way for the user. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the consent unit may be performed using AI, or may be performed without using AI. For example, the consent unit may input the user's emotion data into generative AI to execute adjustment of the method of obtaining consent. Specifically, the consent unit simultaneously acquires the user's facial expression images (e.g., RGB 3-channel 224×224 pixel facial image tensor), voice data (e.g., 5-second WAV format audio waveform), and biometric sensor data (e.g., heart rate, skin conductance values, etc.), and inputs these into an emotion estimation AI model. The AI model is constructed by combining, for example, a multimodal Transformer, ResNet-based facial expression recognition CNN, and RNN for voice emotion classification. Examples of AI input include “User A's smiling image+normal heart rate+calm voice” and “User B's frowning image+high-stress heart rate+tense voice.” The AI output consists of structured data such as “emotion label (e.g., relaxed, stressed, busy)” and “emotion score (e.g., relaxation level 0.85, stress level 0.72).” The consent unit receives the output of the emotion estimation AI model and provides prompts such as “detailed explanation,” “concise explanation,” or “explanation with only key points” to a consent explanation generation AI (e.g., text generation LLM or summary generation model), automatically switching the method of obtaining consent. For example, for a user in a relaxed state, the system generates a detailed explanation such as “This system anonymizes oral images and diagnosis data for health management purposes. Additional consent will be obtained when providing data to third parties,” while for a user in a stressed state, it generates a concise explanation such as “Do you consent to the use of diagnosis data?” Examples of AI output include “detailed consent explanation,” “summary consent comment,” and “instructions for obtaining consent.” In subsequent processing, the consent unit displays the generated explanation on the user interface and links the consent acquisition history to the recording unit. Unlike conventional uniform consent explanations or manual adjustment of explanations, the present consent unit combines multidimensional emotion estimation and optimization of the method of obtaining consent using AI to automatically generate easily understandable consent acquisition for each user, prevent excessive or insufficient explanations, and improve the consent acquisition rate, thereby achieving essential improvements in computer technology. Technical effects include personalized consent acquisition process, optimized explanations, improved user satisfaction, and automated legal compliance. Specific application fields include corporate health management consent acquisition, electronic consent in home healthcare, data usage consent by insurance companies, and informed consent in research institutions.

The consent unit can provide detailed explanations to the user at the time of obtaining consent to deepen understanding. For example, at the time of obtaining consent, the consent unit provides detailed explanations about the diagnosis content and data usage. The consent unit can also carefully answer the user's questions at the time of obtaining consent to deepen understanding. Furthermore, the consent unit can provide detailed explanations about the method of sharing diagnosis results at the time of obtaining consent. By providing detailed explanations, the user's understanding can be deepened and appropriate consent can be obtained. Some or all of the above-described processes in the consent unit may be performed using AI, or may be performed without using AI. For example, the consent unit may input explanation content into an AI model to execute detailed explanations. Specifically, the consent unit collects text data such as diagnosis content, purpose of data usage, scope of third-party provision, data retention period, and user questions, and inputs these into an explanation generation AI model (e.g., text generation LLM, FAQ response model, summary generation model). Examples of AI input include “Diagnosis content: oral image analysis, purpose of data usage: health management, question: Where is the data stored?” and “Method of sharing diagnosis results: anonymized data transmission to specialists, question: Is it shared with family?” The AI output consists of structured data such as “detailed explanation text (e.g., ‘This system anonymizes oral images and diagnosis data and uses them only for health management purposes. Additional consent will be obtained when providing data to third parties. Data is encrypted with AES-256 and stored in the cloud’),” “FAQ response (e.g., ‘Sharing with family is performed only with user consent’),” and “summary of explanation content.” For example, outputs such as “detailed explanation of diagnosis content, purpose of use, retention period, and scope of third-party provision” and “individual FAQ responses to user questions” can be obtained. In subsequent processing, the consent unit displays detailed explanations and FAQs on the user interface based on the AI output, and links confirmation of user understanding and consent acquisition history to the recording unit. Unlike conventional uniform explanations or manual question responses, the present consent unit automates generation of explanation content, FAQ responses, and explanation summaries using AI, thereby achieving essential improvements in computer technology such as improved quality of explanations, increased user understanding, and improved consent acquisition rate. Technical effects include personalized explanation content, prevention of excessive or insufficient explanations, improved user satisfaction, and automated legal compliance. Specific application fields include corporate health management consent acquisition, electronic consent in home healthcare, data usage consent by insurance companies, and informed consent in research institutions.

The consent unit can record the user's consent history at the time of obtaining consent so that it can be referenced later. For example, the consent unit records the user's consent history so that it can be referenced later. The consent unit can also save the user's consent history in a database at the time of obtaining consent. Furthermore, the consent unit can record the user's consent history so that it can be referenced as needed. By recording the consent history, it can be referenced later and appropriate consent management can be performed. Some or all of the above-described processes in the consent unit may be performed using AI, or may be performed without using AI. For example, the consent unit may input consent history recording into an AI model to execute recording. Specifically, the consent unit records structured data such as user ID, consent acquisition date and time, consent content (e.g., scope of data usage, permission for third-party provision, retention period, etc.), explanation content at the time of consent acquisition, and user's response content. When using AI, the consent unit inputs consent history data, user attributes, and past consent change history into an AI model (e.g., Isolation Forest for anomaly detection, Transformer for history summarization, consent change prediction model) to automatically execute recording, summarization, anomaly detection, and change prediction of consent history. Examples of AI input include “User A: consent obtained on 2024 Jun. 1, content: permission to use diagnosis data, no third-party provision” and “User B: consent changed on 2024 Jun. 10, content: permission for family sharing.” The AI output consists of structured data such as “consent history recording instructions,” “summary of consent content,” and “consent change alert.” For example, outputs such as “record consent history in chronological order,” “notify administrator of summary of consent content,” and “alert for abnormal consent changes” can be obtained. In subsequent processing, the consent unit saves consent history in a database based on the AI output, and makes it available for reference by administrators or the user as needed. Unlike conventional paper-based or manual consent history management, the present consent unit automates recording, summarization, and anomaly detection of consent history using AI, thereby achieving essential improvements in computer technology such as more efficient consent management, strengthened legal compliance, and increased user trust. Technical effects include visualization of consent history, tracking of consent changes, more efficient audit response, and enhanced system operation efficiency. Specific application fields include corporate health management consent history management, electronic consent recording in home healthcare, consent change management by insurance companies, and informed consent history management in research institutions.

The consent unit can estimate the user's emotion and adjust the timing of obtaining consent based on the estimated emotional state of the user. For example, if the user is relaxed, the consent unit flexibly adjusts the timing of obtaining consent. If the user is feeling stressed, the consent unit can shorten the timing of obtaining consent. Furthermore, if the user is busy, the consent unit can adjust the timing of obtaining consent according to the user's schedule. By adjusting the timing of obtaining consent based on the user's emotion, consent can be obtained at the optimal timing for the user. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the consent unit may be performed using AI, or may be performed without using AI. For example, the consent unit may input the user's emotion data into generative AI to execute adjustment of the timing of obtaining consent. Specifically, the consent unit simultaneously acquires the user's facial expression images, voice data, biometric sensor data (e.g., heart rate, skin conductance values, etc.), and user's schedule data (e.g., work calendar, private appointments, etc.), and inputs these into an emotion estimation AI model and scheduling AI model. The AI model is constructed by combining, for example, a multimodal Transformer, LSTM-based time-series emotion estimation model, and calendar optimization algorithm. Examples of AI input include “User A's smiling image+normal heart rate+available time slot” and “User B's frowning image+high-stress heart rate+meeting schedule.” The AI output consists of structured data such as “emotion state label (e.g., relaxed, stressed, busy),” “recommended timing for obtaining consent (e.g., 5 minutes later, immediate, after work),” and “reason for timing adjustment.” For example, outputs such as “flexible consent acquisition possible for User A due to relaxed state” and “immediate consent acquisition recommended for User B due to stressed state” can be obtained. In subsequent processing, the consent unit automatically sends consent acquisition requests at the optimal timing by linking with the calendar API and notification functions of the user terminal based on the AI output. Unlike conventional fixed-time consent acquisition or manual timing adjustment, the present consent unit combines integrated analysis of emotional state and schedule and optimization of consent acquisition timing using AI to improve the consent acquisition rate, reduce user burden, and prevent excessive or insufficient explanations, thereby achieving essential improvements in computer technology. Technical effects include personalized consent acquisition timing, improved user experience, enhanced system operation efficiency, and automated legal compliance. Specific application fields include corporate health management consent acquisition, electronic consent in home healthcare, data usage consent by insurance companies, and informed consent in research institutions.

The consent unit can provide explanations according to the user's language and culture at the time of obtaining consent. For example, the consent unit provides explanations according to the user's language at the time of obtaining consent. The consent unit can also provide explanations with consideration for the user's culture at the time of obtaining consent. Furthermore, the consent unit can customize explanations according to the user's language and culture at the time of obtaining consent. By providing explanations according to the user's language and culture, easily understandable explanations can be provided and appropriate consent can be obtained. Some or all of the above-described processes in the consent unit may be performed using AI, or may be performed without using AI. For example, the consent unit may input language and culture data into an AI model to execute customization of explanations. Specifically, the consent unit collects the user's language settings (e.g., Japanese, English, Chinese, etc.), cultural attributes (e.g., religion, region, customs, etc.), past consent acquisition history, and user questions, and inputs these into a multilingual AI model (e.g., multilingual text generation LLM, culture-adaptive summary model). Examples of AI input include “Language: English, Culture: Islamic region, Question: Where is the data stored?” and “Language: Japanese, Culture: elderly, Explanation content: use of diagnosis data.” The AI output consists of structured data such as “multilingual explanation text (e.g., ‘This system anonymizes your oral health data . . . ’),” “culture-adaptive explanation (e.g., specifying religious considerations),” and “summary of explanation content.” For example, outputs such as “generate detailed explanation in English” and “specify religious restrictions on data usage” can be obtained. In subsequent processing, the consent unit displays multilingual and culture-adaptive explanations on the user interface based on the AI output, and links confirmation of user understanding and consent acquisition history to the recording unit. Unlike conventional single-language or non-culture-adaptive explanations or manual translation, the present consent unit automates generation of multilingual and culture-adaptive explanations using AI, thereby achieving essential improvements in computer technology such as improved quality of explanations, increased user understanding, and improved consent acquisition rate. Technical effects include personalized explanation content, enhanced global adaptability, improved user satisfaction, and automated legal compliance. Specific application fields include consent acquisition for multinational employee health management in companies, multilingual electronic consent in home healthcare, global data usage consent by insurance companies, and international joint research informed consent in research institutions.

The consent unit can request consent from the user's family or medical personnel at the time of obtaining consent. For example, the consent unit requests consent from the user's family and explains sharing of diagnosis results. The consent unit can also request consent from the user's medical personnel and explain usage of diagnosis results. Furthermore, the consent unit can request consent from both the user's family and medical personnel to support comprehensive health management. By requesting consent from the user's family or medical personnel, comprehensive health management can be supported. Some or all of the above-described processes in the consent unit may be performed using AI, or may be performed without using AI. For example, the consent unit may input consent content into an AI model to execute consent acquisition from family or medical personnel. Specifically, the consent unit collects family contact information and medical personnel contact information (e.g., email addresses, SMS numbers, medical institution API endpoints) registered in advance by the user, consent content (e.g., scope of sharing diagnosis results, purpose of use, etc.), and attributes of family and medical personnel (e.g., age, occupation, relationship, etc.), and inputs these into a consent acquisition AI model (e.g., text generation LLM, summary generation model, relationship estimation module). Examples of AI input include “User A's diagnosis result+family contact list+consent content: sharing permitted” and “User B's diagnosis result+primary physician API endpoint+consent content: usage permitted.” The AI output consists of structured data such as “consent explanation text for family (e.g., ‘We will share ○○'s diagnosis result. Do you consent?’),” “consent explanation for medical personnel (e.g., ‘Diagnosis data will be used for treatment purposes. Please provide consent’),” and “instructions for obtaining consent.” For example, outputs such as “generate concise consent explanation for family, detailed consent explanation for medical personnel,” and “simultaneous consent acquisition for both in emergencies” can be obtained. In subsequent processing, the consent unit automatically sends consent acquisition requests to family or medical personnel devices or systems based on the AI output, and records and manages consent acquisition history and response status. Unlike conventional consent acquisition only from the user or manual explanation to family or medical personnel, the present consent unit achieves improvements in computer technology by generating consent explanations for each recipient, automating transmission, and managing history using AI, resulting in more efficient sharing of health information, rapid cooperation in emergencies, and strengthened support systems for family and medical personnel. Technical effects include multidimensional cooperation in consent acquisition, prevention of important information transmission failures, promotion of cooperation among users, family, and medical personnel, and enhanced system operation efficiency. Specific application fields include family cooperation consent in employee health management in companies, family and primary physician consent for home healthcare patients, guardian and medical institution cooperation consent at schools and elderly facilities, and health promotion programs by insurance companies.

The proposal unit can estimate the user's emotion and adjust the content of suggestions based on the estimated emotional state of the user. For example, if the user is relaxed, the proposal unit provides detailed suggestion content. If the user is feeling stressed, the proposal unit can provide concise suggestion content. Furthermore, if the user is busy, the proposal unit can provide suggestion content that focuses on key points. By adjusting the content of suggestions based on the user's emotion, the user can receive suggestions with optimal content. Emotion estimation is realized using an emotion estimation function, for example, by employing an emotion engine or generative AI. Generative AI may include text generation AI (e.g., LLM) or multimodal generative AI, but is not limited to these examples. Some or all of the above-described processes in the proposal unit may be performed using AI, or may be performed without using AI. For example, the proposal unit may input the user's emotion data into generative AI to execute adjustment of suggestion content. Specifically, the proposal unit simultaneously acquires the user's facial expression images (e.g., RGB 3-channel 224×224 pixel facial image tensor), voice data (e.g., 5-second WAV format audio waveform), and biometric sensor data (e.g., heart rate, skin conductance values, etc.), and inputs these into an emotion estimation AI model. The proposal unit uses an AI model combining a multimodal Transformer, ResNet-based facial expression recognition CNN, and RNN for voice emotion classification to estimate the user's emotional state with high accuracy. Examples of AI input include “User A's smiling image+normal heart rate+calm voice” and “User B's frowning image+high-stress heart rate+tense voice.” The AI output consists of structured data such as “emotion label (e.g., relaxed, stressed, busy)” and “emotion score (e.g., relaxation level 0.85, stress level 0.72).” The proposal unit receives the output of the emotion estimation AI model and provides prompts such as “detailed suggestion content,” “concise suggestion content,” or “suggestion content with only key points” to a suggestion content generation AI (e.g., text generation LLM or summary generation model), automatically switching the expression style of suggestion content. For example, for a user in a relaxed state, the system generates a detailed explanation such as “Suspected caries in lower right molar No. 6 (score 0.92), recommended treatment: resin filling, self-care advice: brush teeth and use floss after every meal,” while for a user in a stressed state, it generates a concise explanation such as “Suspected caries. Dental visit recommended.” Examples of AI output include “detailed suggestion report,” “summary suggestion comment,” and “suggestion content expression style instructions.” In subsequent processing, the proposal unit links the generated suggestion content to the notification unit and recording unit, and delivers it to the user terminal or management system in the optimal format. Unlike conventional uniform suggestion content or manual adjustment of explanations, the present proposal unit combines multidimensional emotion estimation and suggestion content expression optimization using AI to automatically generate easily understandable suggestion content for each user, prevent excessive or insufficient explanations, and improve user experience, thereby achieving essential improvements in computer technology. Technical effects include personalized suggestion content, optimized explanations, improved user satisfaction, and enhanced system operation efficiency. Specific application fields include stress-considerate suggestion notifications for employee health management in companies, burden-reducing suggestion reports for home healthcare patients, optimized explanations for group health suggestions at schools and elderly facilities, and health promotion programs by insurance companies.

The proposal unit can refer to the user's past diagnosis data at the time of suggestion to propose the optimal treatment method. For example, the proposal unit refers to the user's past diagnosis data and proposes the optimal treatment method. Furthermore, the proposal unit can customize the treatment method based on the user's past diagnosis data. Additionally, the proposal unit can comprehensively refer to the user's past diagnosis data to propose the optimal treatment method. By referring to past diagnosis data, the optimal treatment method can be proposed. Some or all of the above-described processes in the proposal unit may be performed using AI or without using AI. For example, the proposal unit inputs past diagnosis data into an AI model to execute the proposal of treatment methods. Specifically, the proposal unit inputs time-series diagnosis result data recorded for each user (e.g., abnormality detection labels, coordinates of abnormal areas, diagnosis confidence scores, treatment history, lifestyle and dietary data, etc.) as time-series tensors or history vectors into the AI model. The proposal unit uses time-series recurrent neural networks such as LSTM or GRU, autoregressive models, and anomaly detection algorithms (e.g., Isolation Forest) to perform comparison with past data, trend analysis, and anomaly detection, thereby optimizing treatment methods. Examples of AI input include “diagnosis score array for user A over the past 12 months,” “treatment history vector for user B,” and “history of abnormal area coordinates by diagnosis date.” The AI output consists of structured data such as “recommended treatment methods (e.g., resin filling, periodontal pocket cleaning, follow-up observation),” “treatment priority score (e.g., 0.92),” and “treatment customization instructions.” For example, outputs such as “resin filling recommended due to increased risk of cavities over the past 6 months” and “continued follow-up observation due to lifestyle improvement” can be obtained. The proposal unit generates the content of treatment suggestions based on the AI output and cooperates with the notification unit or recording unit to deliver the information to the user terminal or management system. Unlike conventional simple treatment suggestions or manual reference to history, the proposal unit realizes time-series data analysis, anomaly detection, and optimization of treatment suggestions using AI, thereby improving the accuracy of treatment suggestions, enabling early detection of anomalies, and reducing user burden, which leads to improvements in computer technology. Technical effects include personalization of treatment suggestions, optimization of treatment plans, enhancement of user experience, and efficient utilization of medical resources. Specific application fields include treatment suggestions for employee health management in companies, follow-up observation-type treatment suggestions for home medical patients, health promotion programs by insurance companies, and analysis of treatment effects in research institutions.

The proposal unit can provide specific advice at the time of suggestion based on the user's lifestyle and dietary habits. For example, the proposal unit considers the user's lifestyle and provides specific advice. The proposal unit can also consider the user's dietary habits and provide specific advice. Furthermore, the proposal unit can comprehensively consider both the user's lifestyle and dietary habits to provide specific advice. By providing specific advice based on lifestyle and dietary habits, the proposal unit can offer optimal advice to the user. Some or all of the above-described processes in the proposal unit may be performed using AI or without using AI. For example, the proposal unit inputs lifestyle and dietary data into an AI model to execute the provision of advice. Specifically, the proposal unit vectorizes meal records recorded by the user (e.g., daily meal records, intake amounts of carbohydrates, fats, vegetables, etc.) and lifestyle data (e.g., frequency of tooth brushing, smoking history, drinking habits, sleep duration, amount of exercise, etc.), and inputs them into an AI model (e.g., multimodal Transformer, gradient boosting decision tree, logistic regression, etc.). Examples of AI input include “User A: brushes teeth three times a day, frequent snacking, low vegetable intake” and “User B: has a smoking history, insufficient sleep, no exercise habit.” The AI output consists of structured data such as “recommended advice (e.g., reduce snacking frequency, increase vegetable intake, recommend quitting smoking),” “diagnosis score after risk adjustment (e.g., cavity risk 0.85→0.92),” and “comment on reason for adjustment (e.g., increased cavity risk due to increased snacking frequency).” The proposal unit integrates the AI output with image diagnosis results and bacterial risk diagnosis results to generate the final advice content. As a subsequent process, the proposal unit cooperates with the notification unit or recording unit to deliver the generated advice to the user terminal or management system in the optimal format. Unlike conventional simple advice or manual consideration of lifestyle habits, the proposal unit realizes multidimensional data integration, risk adjustment, and optimization of advice using AI, thereby improving the accuracy of advice, enabling early detection of anomalies, and reducing user burden, which leads to improvements in computer technology. Technical effects include personalization of advice, improvement of risk assessment accuracy, promotion of user behavioral change, and efficient utilization of medical resources. Specific application fields include employee health management in companies, lifestyle guidance in home medical care, health promotion programs by insurance companies, and health education in schools and elderly care facilities.

The proposal unit can estimate the user's emotion and determine the priority of suggestions based on the estimated emotion. For example, if the user is relaxed, the proposal unit lowers the priority of suggestions. If the user is feeling stressed, the proposal unit can increase the priority of suggestions. Furthermore, if the user is busy, the proposal unit can adjust the priority of suggestions according to the user's schedule. By determining the priority of suggestions based on the user's emotion, the user can receive suggestions in the optimal order. Emotion estimation is realized, for example, by using an emotion engine or generative AI with emotion estimation functions. Generative AI may include text generation AI (e.g., LLM) or multimodal generative AI, but is not limited to these examples. Some or all of the above-described processes in the proposal unit may be performed using AI or without using AI. For example, the proposal unit can input the user's emotion data into generative AI and have the generative AI determine the priority of suggestions. Specifically, the proposal unit simultaneously acquires the user's facial expression images, voice data, and biometric sensor data (e.g., heart rate, skin conductance response, etc.), and inputs these into an emotion estimation AI model. The AI model is constructed by combining multimodal Transformers, ResNet-based facial expression recognition CNNs, and RNNs for voice emotion classification. Examples of AI input include “User A's smiling image+normal heart rate+calm voice” and “User B's frowning image+high-stress heart rate+tense voice.” The AI output consists of structured data such as “emotion label (e.g., relaxed, stressed, busy),” “emotion score (e.g., relaxation level 0.85, stress level 0.72),” and “recommended suggestion priority (e.g., high/medium/low).” The proposal unit automatically adjusts the order and content of suggestions for transmission based on the AI output and delivers them to the user terminal or management system in the optimal format. Unlike conventional uniform suggestion priorities or manual urgency determination, the proposal unit realizes integrated analysis of emotional state data and optimization of suggestion priorities using AI, thereby preventing the oversight of important suggestions, reducing user burden, and improving the efficiency of suggestion operations, which leads to improvements in computer technology. Technical effects include personalization of suggestion priorities, immediate transmission of important suggestions, enhancement of user experience, and improvement of system operation efficiency. Specific application fields include health management suggestions for company employees, urgent suggestions for home medical patients, health promotion program suggestions by insurance companies, and health suggestions in schools and elderly care facilities.

The proposal unit can customize the content of suggestions at the time of suggestion according to the user's health condition. For example, if the user's health condition is deteriorating, the proposal unit proposes specific treatment methods. If the user's health condition is stable, the proposal unit can provide preventive advice. Furthermore, the proposal unit can comprehensively consider the user's health condition to customize the content of suggestions. By customizing the content of suggestions according to the health condition, the proposal unit can provide the user with the optimal suggestion content. Some or all of the above-described processes in the proposal unit may be performed using AI or without using AI. For example, the proposal unit inputs health condition data into an AI model to execute customization of suggestion content. Specifically, the proposal unit collects health condition data output from the diagnosis unit or recording unit (e.g., abnormality detection labels, coordinates of abnormal areas, diagnosis confidence scores, lifestyle and dietary data, etc.) and inputs these into the AI model. The AI model uses gradient boosting decision trees, logistic regression, multimodal Transformers, etc., to score the severity and urgency of the health condition and automatically customize the suggestion content. Examples of AI input include “User A: suspected cavity (score 0.92), signs of periodontal disease (score 0.78), lifestyle: smoking history” and “User B: stable health condition, low abnormality score.” The AI output consists of structured data such as “recommended suggestion content (e.g., resin filling recommended, enhanced self-care, follow-up observation),” “urgency label for suggestion content,” and “suggestion transmission timing instructions.” The proposal unit generates the suggestion content based on the AI output and delivers it to the user terminal or management system in the optimal format. Unlike conventional uniform suggestion content or manual adjustment of content, the proposal unit realizes integrated analysis of health condition data and optimization of suggestion content using AI, thereby enabling personalization of suggestion content, immediate transmission of important suggestions, enhancement of user experience, and improvement of system operation efficiency, which leads to improvements in computer technology. Technical effects include personalization of suggestion content, optimization of explanations, improvement of user satisfaction, and improvement of system operation efficiency. Specific application fields include health management suggestions for company employees, treatment suggestions for home medical patients, health promotion program suggestions by insurance companies, and health suggestions in schools and elderly care facilities.

The proposal unit can share the content of suggestions with the user's family or medical personnel at the time of suggestion. For example, the proposal unit shares the content of suggestions with the user's family to support health management. The proposal unit can also share the content of suggestions with the user's medical personnel to discuss treatment methods. Furthermore, the proposal unit can share the content of suggestions with both the user's family and medical personnel to support comprehensive health management. By sharing the content of suggestions with family and medical personnel, comprehensive health management can be supported. Some or all of the above-described processes in the proposal unit may be performed using AI or without using AI. For example, the proposal unit inputs the content of suggestions into an AI model to execute sharing with family or medical personnel. Specifically, the proposal unit collects suggestion content data output from the diagnosis unit or recording unit (e.g., treatment methods, self-care advice, lifestyle improvement suggestions, etc.) and manages them in association with contact information for family members or medical personnel registered in advance by the user (e.g., email addresses, SMS numbers, medical institution API endpoints, etc.). The proposal unit automatically optimizes the granularity and expression of suggestion content for each recipient using an AI model (e.g., multimodal Transformer, gradient boosting decision tree, user attribute/relationship estimation module, etc.) to generate suggestion content such as “detailed suggestion report,” “summary suggestion,” and “emergency alert” for each recipient. Examples of AI input include “User A's suggestion content+family contact list,” “User B's suggestion content+primary physician API endpoint,” and “User C's suggestion content+both family and medical personnel.” The AI output consists of structured data such as “suggestion content for family (e.g., ‘Signs of cavities have been found in ○○'s oral cavity. Please support a medical visit’),” “treatment suggestion data packet for medical personnel (e.g., JSON including treatment methods, diagnosis confidence scores, summary of past history, etc.),” and “instructions for transmission timing and channel (e.g., SMS for family, immediate transmission via API for medical personnel).” As a subsequent process, the proposal unit automatically sends the suggestion content to family or medical personnel devices or systems based on the AI output and records and manages transmission history and read status. Unlike conventional suggestions only to the user or manual contact with family or medical personnel, the proposal unit realizes optimization of content for each recipient, automatic transmission, and history management using AI, thereby improving the efficiency of health information sharing, enabling rapid cooperation in emergencies, and strengthening the support system for family and medical personnel, which leads to improvements in computer technology. Technical effects include multidimensional cooperation in health management, prevention of information transmission omissions, promotion of cooperation among users, family, and medical personnel, and improvement of system operation efficiency. Specific application fields include family cooperation suggestions for employee health management in companies, family and primary physician cooperation suggestions for home medical patients, parent and medical institution cooperation suggestions in schools and elderly care facilities, and health promotion programs by insurance companies.

The recording unit can estimate the user's emotion and adjust the content of records based on the estimated emotion. For example, if the user is relaxed, the recording unit provides detailed record content. If the user is feeling stressed, the recording unit can provide concise record content. Furthermore, if the user is busy, the recording unit can provide record content that focuses on key points. By adjusting the content of records based on the user's emotion, the recording unit can provide the user with the optimal record content. Emotion estimation is realized, for example, by using an emotion engine or generative AI with emotion estimation functions. Generative AI may include text generation AI (e.g., LLM) or multimodal generative AI, but is not limited to these examples. Some or all of the above-described processes in the recording unit may be performed using AI or without using AI. For example, the recording unit can input the user's emotion data into generative AI and have the generative AI adjust the content of records.

The recording unit can analyze changes in health condition by comparing the user's past diagnosis data at the time of recording. For example, the recording unit compares the user's past diagnosis data with current diagnosis data to analyze changes in health condition. The recording unit can also refer to the user's past diagnosis data and record changes in health condition. Furthermore, the recording unit can comprehensively compare the user's past and current diagnosis data to analyze changes in health condition. By comparing with past diagnosis data, changes in health condition can be analyzed. Some or all of the above-described processes in the recording unit may be performed using AI or without using AI. For example, the recording unit inputs past diagnosis data into an AI model to execute analysis of changes in health condition.

The recording unit can also record the user's lifestyle and dietary data at the time of recording. For example, the recording unit records the user's lifestyle data and analyzes changes in health condition. The recording unit can also record the user's dietary data and analyze changes in health condition. Furthermore, the recording unit can comprehensively record both the user's lifestyle and dietary data and analyze changes in health condition. By recording lifestyle and dietary data together, changes in health condition can be comprehensively analyzed. Some or all of the above-described processes in the recording unit may be performed using AI or without using AI. For example, the recording unit inputs lifestyle and dietary data into an AI model to execute recording.

The recording unit can estimate the user's emotion and adjust the frequency of recording based on the estimated emotion. For example, if the user is relaxed, the recording unit reduces the frequency of recording. If the user is feeling stressed, the recording unit can increase the frequency of recording. Furthermore, if the user is busy, the recording unit can adjust the frequency of recording according to the user's schedule. By adjusting the frequency of recording based on the user's emotion, the recording unit can record at the optimal frequency for the user. Emotion estimation is realized, for example, by using an emotion engine or generative AI with emotion estimation functions. Generative AI may include text generation AI (e.g., LLM) or multimodal generative AI, but is not limited to these examples. Some or all of the above-described processes in the recording unit may be performed using AI or without using AI. For example, the recording unit can input the user's emotion data into generative AI and have the generative AI adjust the frequency of recording.

The recording unit can store the user's health data in the cloud at the time of recording so that it can be accessed at any time. For example, the recording unit stores the user's health data in the cloud so that it can be accessed at any time. The recording unit can also store the user's health data in the cloud to prevent data loss. Furthermore, the recording unit can store the user's health data in the cloud so that it can be accessed from multiple devices. By storing health data in the cloud, it can be accessed at any time and data loss can be prevented. Some or all of the above-described processes in the recording unit may be performed using AI or without using AI. For example, the recording unit inputs instructions for cloud storage into an AI model to execute data storage.

The recording unit can anonymize the user's health data at the time of recording and provide it to research institutions. For example, the recording unit anonymizes the user's health data and provides it to research institutions. The recording unit can also anonymize the user's health data and use it for research purposes. Furthermore, the recording unit can anonymize the user's health data and provide it to third parties. By anonymizing health data and providing it to research institutions, privacy can be protected while contributing to research. Some or all of the above-described processes in the recording unit may be performed using AI or without using AI. For example, the recording unit inputs the use of anonymization technology into an AI model to execute data anonymization.

The sharing unit can estimate the user's emotion and adjust the content of sharing based on the estimated emotion. For example, if the user is relaxed, the sharing unit provides detailed sharing content. If the user is feeling stressed, the sharing unit can provide concise sharing content. Furthermore, if the user is busy, the sharing unit can provide sharing content that focuses on key points. By adjusting the content of sharing based on the user's emotion, the sharing unit can share information in the optimal format for the user. Emotion estimation is realized, for example, by using an emotion engine or generative AI with emotion estimation functions. Generative AI may include text generation AI (e.g., LLM) or multimodal generative AI, but is not limited to these examples. Some or all of the above-described processes in the sharing unit may be performed using AI or without using AI. For example, the sharing unit can input the user's emotion data into generative AI and have the generative AI adjust the content of sharing.

The sharing unit can provide detailed diagnosis data to a professional dentist at the time of sharing. For example, the sharing unit provides detailed diagnosis data to a professional dentist. The sharing unit can also provide detailed explanations of diagnosis results to a professional dentist. Furthermore, the sharing unit can provide diagnosis data to a professional dentist and request expert opinions. By providing detailed diagnosis data to a professional dentist, expert opinions can be obtained. Some or all of the above-described processes in the sharing unit may be performed using AI or without using AI. For example, the sharing unit inputs the provision of diagnosis data into an AI model to execute data provision.

The sharing unit can also provide the user's past diagnosis data at the time of sharing to obtain comprehensive opinions. For example, the sharing unit provides the user's past diagnosis data together to obtain comprehensive opinions. The sharing unit can also refer to the user's past diagnosis data and request opinions from a professional dentist. Furthermore, the sharing unit can provide the user's past diagnosis data to perform comprehensive diagnosis. By providing past diagnosis data together, comprehensive opinions can be obtained. Some or all of the above-described processes in the sharing unit may be performed using AI or without using AI. For example, the sharing unit inputs the provision of past diagnosis data into an AI model to execute data provision.

The sharing unit can estimate the user's emotion and adjust the timing of sharing based on the estimated emotion. For example, if the user is relaxed, the sharing unit flexibly adjusts the timing of sharing. If the user is feeling stressed, the sharing unit can shorten the timing of sharing. Furthermore, if the user is busy, the sharing unit can adjust the timing of sharing according to the user's schedule. By adjusting the timing of sharing based on the user's emotion, the sharing unit can share information at the optimal timing for the user. Emotion estimation is realized, for example, by using an emotion engine or generative AI with emotion estimation functions. Generative AI may include text generation AI (e.g., LLM) or multimodal generative AI, but is not limited to these examples. Some or all of the above-described processes in the sharing unit may be performed using AI or without using AI. For example, the sharing unit can input the user's emotion data into generative AI and have the generative AI adjust the timing of sharing.

The sharing unit can introduce encryption technology at the time of sharing to protect the user's privacy. For example, the sharing unit encrypts diagnosis data and provides it to a professional dentist. The sharing unit can also use encryption technology when transmitting data to protect the user's privacy. Furthermore, the sharing unit can encrypt the user's diagnosis data to prevent access by third parties. By introducing encryption technology to protect privacy, data security can be ensured. Some or all of the above-described processes in the sharing unit may be performed using AI or without using AI. For example, the sharing unit inputs the use of encryption technology into an AI model to execute data encryption.

The sharing unit can anonymize the user's health data at the time of sharing and provide it to a professional dentist. For example, the sharing unit anonymizes the user's health data and provides it to a professional dentist. The sharing unit can also anonymize health data and transmit it to protect the user's privacy. Furthermore, the sharing unit can anonymize the user's health data and provide it to a professional dentist to obtain comprehensive opinions. By anonymizing health data and providing it to a professional dentist, privacy can be protected while obtaining expert opinions. Some or all of the above-described processes in the sharing unit may be performed using AI or without using AI. For example, the sharing unit inputs the use of anonymization technology into an AI model to execute data anonymization.

The reminder unit can estimate the user's emotion and adjust the content of reminders based on the estimated emotion. For example, if the user is relaxed, the reminder unit provides detailed reminder content. If the user is feeling stressed, the reminder unit can provide concise reminder content. Furthermore, if the user is busy, the reminder unit can provide reminder content that focuses on key points. By adjusting the content of reminders based on the user's emotion, the reminder unit can deliver reminders in the optimal format for the user. Emotion estimation is realized, for example, by using an emotion engine or generative AI with emotion estimation functions. Generative AI may include text generation AI (e.g., LLM) or multimodal generative AI, but is not limited to these examples. Some or all of the above-described processes in the reminder unit may be performed using AI or without using AI. For example, the reminder unit can input the user's emotion data into generative AI and have the generative AI adjust the content of reminders.

The reminder unit can select the optimal timing for sending reminders at the time of reminder transmission by considering the user's schedule. For example, the reminder unit refers to the user's schedule and selects the optimal timing for sending reminders. The reminder unit can also adjust the timing of reminder transmission according to the user's schedule. Furthermore, the reminder unit can comprehensively consider the user's schedule to select the optimal timing for sending reminders. By considering the schedule and selecting the timing for transmission, the reminder unit can deliver reminders to the user at the optimal timing. Some or all of the above-described processes in the reminder unit may be performed using AI or without using AI. For example, the reminder unit inputs schedule data into an AI model to execute selection of transmission timing.

The reminder unit can select a transmission method (email, SMS, app notification, etc.) according to the user's preferences at the time of reminder transmission. For example, if the user prefers email, the reminder unit sends reminders by email. If the user prefers SMS, the reminder unit can send reminders by SMS. Furthermore, if the user prefers app notifications, the reminder unit can send reminders by app notification. By selecting the transmission method according to the user's preferences, the reminder unit can deliver reminders in the way that is easiest for the user to receive. Some or all of the above-described processes in the reminder unit may be performed using AI or without using AI. For example, the reminder unit inputs the selection of transmission method into an AI model to execute transmission.

The reminder unit can estimate the user's emotion and adjust the frequency of reminders based on the estimated emotional state of the user. For example, if the user is relaxed, the reminder unit reduces the frequency of reminders. If the user is feeling stressed, the reminder unit can increase the frequency of reminders. Furthermore, if the user is busy, the reminder unit can adjust the frequency of reminders according to the user's schedule. By adjusting the frequency of reminders based on the user's emotion, the user can receive reminders at the optimal frequency. Emotion estimation is realized, for example, by using an emotion engine or a generative AI with emotion estimation functions. The generative AI may be a text generation AI (for example, LLM) or a multimodal generative AI, but is not limited to these examples. Some or all of the above-described processes in the reminder unit may be performed using AI or without using AI. For example, the reminder unit can input the user's emotion data to a generative AI and have the generative AI execute the adjustment of reminder frequency.

The reminder unit can customize the content of reminders according to the user's health condition at the time of sending reminders. For example, if the user's health condition is deteriorating, the reminder unit sends a reminder proposing specific treatment methods. If the user's health condition is stable, the reminder unit can also send a reminder providing preventive advice. Furthermore, the reminder unit can comprehensively consider the user's health condition and customize the content of reminders. By customizing the content of reminders according to the user's health condition, the user can receive reminders with the optimal content. Some or all of the above-described processes in the reminder unit may be performed using AI or without using AI. For example, the reminder unit can input health condition data to an AI model and execute customization of the reminder content.

The reminder unit can add a function to send reminders to the user's family or medical personnel at the time of sending reminders. For example, the reminder unit sends reminders to the user's family to support health management. The reminder unit can also send reminders to the user's medical personnel to discuss treatment methods. Furthermore, the reminder unit can send reminders to both the user's family and medical personnel to support comprehensive health management. By sending reminders to the user's family or medical personnel, comprehensive health management can be supported. Some or all of the above-described processes in the reminder unit may be performed using AI or without using AI. For example, the reminder unit can input the reminder content to an AI model and execute sending to the family or medical personnel.

The system according to the embodiment is not limited to the examples described above, and various modifications are possible, for example, as follows.

The diagnosis unit can detect changes in color tone and texture in the oral cavity when analyzing images of the user's oral cavity. For example, it detects changes in color tone in the oral cavity and evaluates the health condition of the gums. It can also detect changes in texture and identify stomatitis or other abnormalities. Furthermore, when analyzing images of the oral cavity, the diagnosis unit can consider temperature and humidity data of the user's oral cavity to improve diagnostic accuracy. By detecting changes in color tone and texture in the oral cavity, more accurate diagnosis becomes possible.

When sending the diagnosis result to the company's health management system, the cooperation unit can consider the user's job content and work status to notify at an appropriate timing. For example, if the user is in an important meeting, the notification is delayed. If the user is on vacation, the notification can be sent after the vacation. Furthermore, the cooperation unit can adjust the content of the notification according to the user's work status. By notifying while considering the user's job content and work status, it is possible to avoid interfering with business operations.

When obtaining the user's consent, the consent unit can adjust the content of explanations according to the user's age and health condition. For example, it provides more detailed explanations to elderly users and concise explanations to younger users. If the user's health condition is deteriorating, it can provide explanations with particular care. Furthermore, the consent unit can repeatedly provide explanations according to the user's level of understanding. By providing explanations according to the user's age and health condition, appropriate consent can be obtained.

Based on the diagnosis result, the proposal unit can propose oral self-care methods to the user. For example, it proposes appropriate tooth brushing methods or flossing methods to the user. It can also provide advice on diet and lifestyle habits to maintain oral health. Furthermore, the proposal unit can recommend the use of specific dental products according to the user's oral condition. By providing specific advice for maintaining oral health in daily life, the user can receive concrete guidance.

When recording the diagnosis result, the recording unit can save images of the user's oral cavity as a 3D model. For example, it saves the condition of the user's oral cavity as a 3D model and compares it with past data. Using the 3D model, changes in the oral cavity can be visually confirmed. Furthermore, the recording unit can save the 3D model in the cloud so that the user can access it at any time. By saving the condition of the oral cavity as a 3D model, more detailed recording and analysis become possible.

When sharing the diagnosis result with a professional dentist, the sharing unit can estimate the user's emotion and adjust the content of sharing based on the estimated emotional state. For example, if the user is relaxed, detailed diagnosis results are provided. If the user is feeling stressed, concise diagnosis results can be provided. Furthermore, if the user is busy, diagnosis results focusing on key points can be provided. By adjusting the content of sharing based on the user's emotion, the diagnosis result can be shared in the optimal manner for the user.

The reminder unit can estimate the user's emotion and adjust the content of reminders based on the estimated emotional state. For example, if the user is relaxed, detailed reminder content is provided. If the user is feeling stressed, concise reminder content can be provided. Furthermore, if the user is busy, reminder content focusing on key points can be provided. By adjusting the content of reminders based on the user's emotion, the user can receive reminders with the optimal content.

The imaging unit can estimate the user's emotion and adjust the timing of imaging based on the estimated emotional state. For example, if the user is relaxed, the timing of imaging is flexibly adjusted, and a timing that is easy for the user to capture images is selected. If the user is feeling stressed, the timing of imaging is shortened, and imaging can be completed quickly. Furthermore, if the user is busy, the timing of imaging can be adjusted according to the user's schedule. By adjusting the timing of imaging based on the user's emotion, the user can capture images in a relaxed manner.

The diagnosis unit can estimate the user's emotion and adjust the manner of expressing the diagnosis result based on the estimated emotional state. For example, if the user is relaxed, detailed diagnosis results are provided. If the user is feeling stressed, concise diagnosis results can be provided. Furthermore, if the user is busy, diagnosis results focusing on key points can be provided. By adjusting the manner of expressing the diagnosis result based on the user's emotion, diagnosis results that are easy for the user to understand can be provided.

The notification unit can estimate the user's emotion and adjust the timing of notification based on the estimated emotional state. For example, if the user is relaxed, the timing of notification is flexibly adjusted. If the user is feeling stressed, the timing of notification can be shortened. Furthermore, if the user is busy, the timing of notification can be adjusted according to the user's schedule. By adjusting the timing of notification based on the user's emotion, the user can receive notifications at the optimal timing.

The following is a brief description of the processing flow of Example of the Embodiment.

Step 1: The imaging unit captures images of the user's oral cavity using the PC's internal camera. The user opens their mouth and captures images facing the internal camera, and these images are sent to the AI.

Step 2: The diagnosis unit analyzes the images sent to the AI and diagnoses the condition of the oral cavity. For example, the AI detects signs of cavities or periodontal disease. Text generation AI (for example, LLM) or generative AI can be used for diagnosis.

Step 3: The notification unit sends the results of the oral check or a prompt to visit a medical institution to the user based on the diagnosis result. For example, a message such as “Signs of cavities detected. Please visit a dental clinic.” is sent by email, SMS, or app notification.

The specific processing unit 290 sends the results of specific processing to the smart device 14. In the smart device 14, the control unit 46A causes the output device 40 to output the results of specific processing. The microphone 38B acquires voice indicating user input in response to the results of specific processing. The control unit 46A sends the voice data indicating user input acquired by the microphone 38B to the data processing device 12. In the data processing device 12, the specific processing unit 290 acquires the voice data.

The data generation model 58 is a so-called generative AI (Artificial Intelligence). An example of the data generation model 58 is a generative AI such as ChatGPT (registered trademark) (Internet search <URL: https://openai.com/blog/chatgpt>). The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

Moreover, the processing by the data processing system 10 described above is executed by the specific processing unit 290 of the data processing device 12 or the control unit 46A of the smart device 14, but it may be executed by both the specific processing unit 290 of the data processing device 12 and the control unit 46A of the smart device 14. Additionally, the specific processing unit 290 of the data processing device 12 acquires or collects necessary information for processing from the smart device 14 or external devices, and the smart device 14 acquires or collects necessary information for processing from the data processing device 12 or external devices.

Each of the plurality of elements including the aforementioned imaging unit, diagnosis unit, notification unit, cooperation unit, consent unit, proposal unit, recording unit, sharing unit, and reminder unit is implemented by at least one of, for example, the smart device 14 and the data processing device 12. For example, the imaging unit captures images of the user's oral cavity using the camera 42 of the smart device 14. The diagnosis unit is implemented, for example, by a specific processing unit 290 of the data processing device 12, and AI analyzes the transmitted images and diagnoses the condition of the oral cavity. The notification unit is implemented, for example, by a control unit 46A of the smart device 14, and sends notifications to the user based on the diagnosis result. The cooperation unit is implemented, for example, by the specific processing unit 290 of the data processing device 12, and transmits the diagnosis result to the company's health management system. The consent unit is implemented, for example, by the control unit 46A of the smart device 14, and obtains the user's consent. The proposal unit is implemented, for example, by the specific processing unit 290 of the data processing device 12, and provides specific advice or treatment suggestions based on the diagnosis result. The recording unit is implemented, for example, by the specific processing unit 290 of the data processing device 12, and records the diagnosis result at regular intervals and compares it with past data. The sharing unit is implemented, for example, by the specific processing unit 290 of the data processing device 12, and shares the diagnosis result with a professional dentist. The reminder unit is implemented, for example, by the control unit 46A of the smart device 14, and sends reminders to receive appropriate treatment. The correspondence between each unit and the device or control unit is not limited to the examples described above and various modifications are possible.

### Second Embodiment

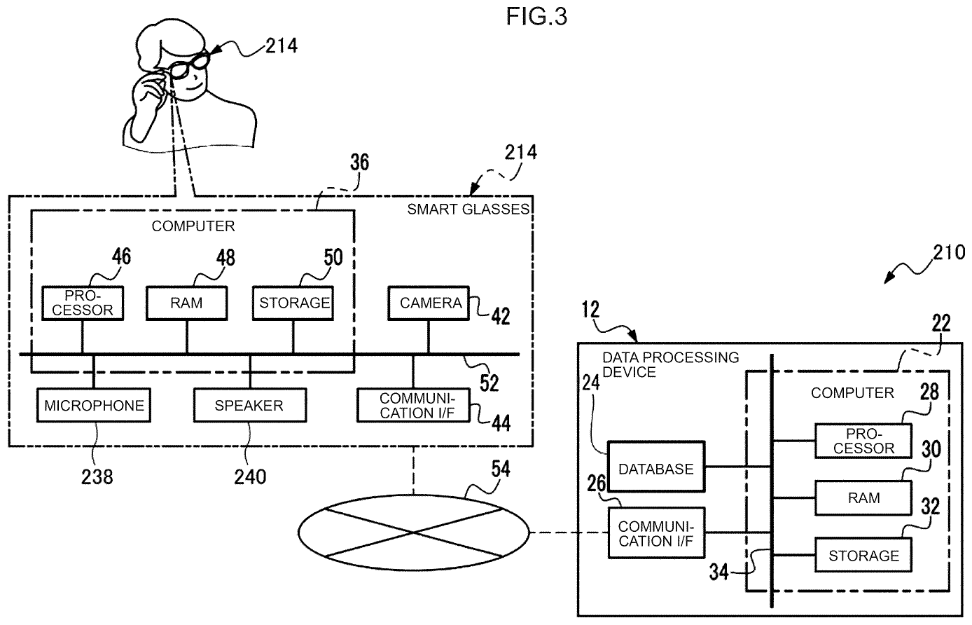

FIG. 3 shows an example configuration of a data processing system 210 according to the second embodiment.

As shown in FIG. 3, the data processing system 210 comprises a data processing device 12 and smart glasses 214. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The smart glasses 214 comprise a computer 36, a microphone 238, a speaker 240, a camera 42, and a communication I/F 44. The computer 36 comprises a processor 46, RAM 48, and storage 50. The processor 46, RAM 48, and storage 50 are connected to a bus 52. The microphone 238, speaker 240, and camera 42 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

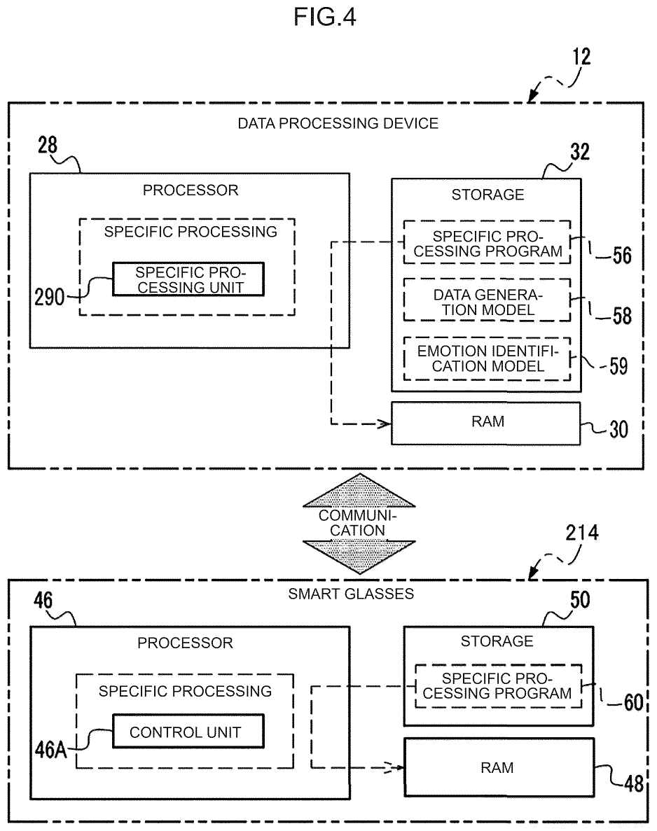

FIG. 4 shows an example of the main functions of the data processing device 12 and smart glasses 214. As shown in FIG. 4, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the smart glasses 214, specific processing is performed by the processor 46. The storage 50 stores a specific processing program 60. The processor 46 reads the specific processing program 60 from the storage 50 and executes it on the RAM 48. The specific processing is realized by the processor 46 operating as a control unit 46A according to the specific processing program 60 executed on the RAM 48. The smart glasses 214 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the smart glasses 214. In the smart glasses 214, the control unit 46A causes the speaker 240 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 210 according to the second embodiment performs the same processing as the data processing system 10 according to the first embodiment. The processing by the data processing system 210 is executed by the specific processing unit 290 of the data processing device 12 or the control unit 46A of the smart glasses 214, but it may be executed by both the specific processing unit 290 of the data processing device 12 and the control unit 46A of the smart glasses 214. Additionally, the specific processing unit 290 of the data processing device 12 acquires or collects necessary information for processing from the smart glasses 214 or external devices, and the smart glasses 214 acquires or collects necessary information for processing from the data processing device 12 or external devices.

Each of the plurality of elements including the aforementioned imaging unit, diagnosis unit, notification unit, cooperation unit, consent unit, proposal unit, recording unit, sharing unit, and reminder unit is implemented by at least one of, for example, the smart glasses 214 and the data processing device 12. For example, the imaging unit captures images of the user's oral cavity using the camera 42 of the smart glasses 214. The diagnosis unit is implemented, for example, by a specific processing unit 290 of the data processing device 12, and AI analyzes the transmitted images and diagnoses the condition of the oral cavity. The notification unit is implemented, for example, by a control unit 46A of the smart glasses 214, and sends notifications to the user based on the diagnosis result. The cooperation unit is implemented, for example, by the specific processing unit 290 of the data processing device 12, and transmits the diagnosis result to the company's health management system. The consent unit is implemented, for example, by the control unit 46A of the smart glasses 214, and obtains the user's consent. The proposal unit is implemented, for example, by the specific processing unit 290 of the data processing device 12, and provides specific advice or treatment suggestions based on the diagnosis result. The recording unit is implemented, for example, by the specific processing unit 290 of the data processing device 12, and records the diagnosis result at regular intervals and compares it with past data. The sharing unit is implemented, for example, by the specific processing unit 290 of the data processing device 12, and shares the diagnosis result with a professional dentist. The reminder unit is implemented, for example, by the control unit 46A of the smart glasses 214, and sends reminders to receive appropriate treatment. The correspondence between each unit and the device or control unit is not limited to the examples described above and various modifications are possible.

### Third Embodiment

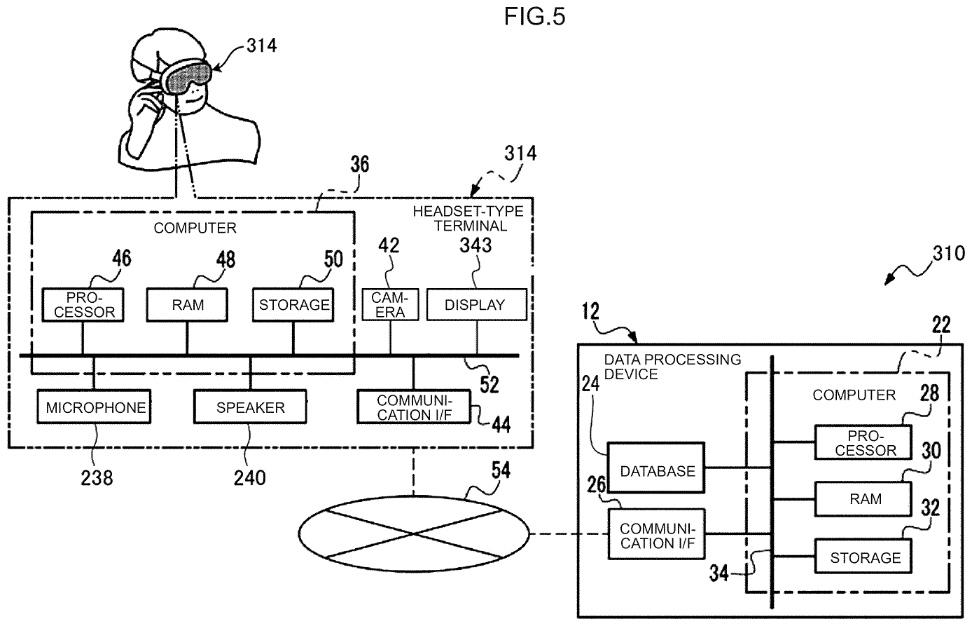

FIG. 5 shows an example configuration of a data processing system 310 according to the third embodiment.

As shown in FIG. 5, the data processing system 310 comprises a data processing device 12 and a headset-type terminal 314. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The headset-type terminal 314 comprises a computer 36, a microphone 238, a speaker 240, a camera 42, a communication I/F 44, and a display 343. The computer 36 comprises a processor 46, RAM 48, and storage 50. The processor 46, RAM 48, and storage 50 are connected to a bus 52. The microphone 238, speaker 240, camera 42, and display 343 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS (Complementary Metal-Oxide-Semiconductor) image sensors or CCD (Charge Coupled Device) image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

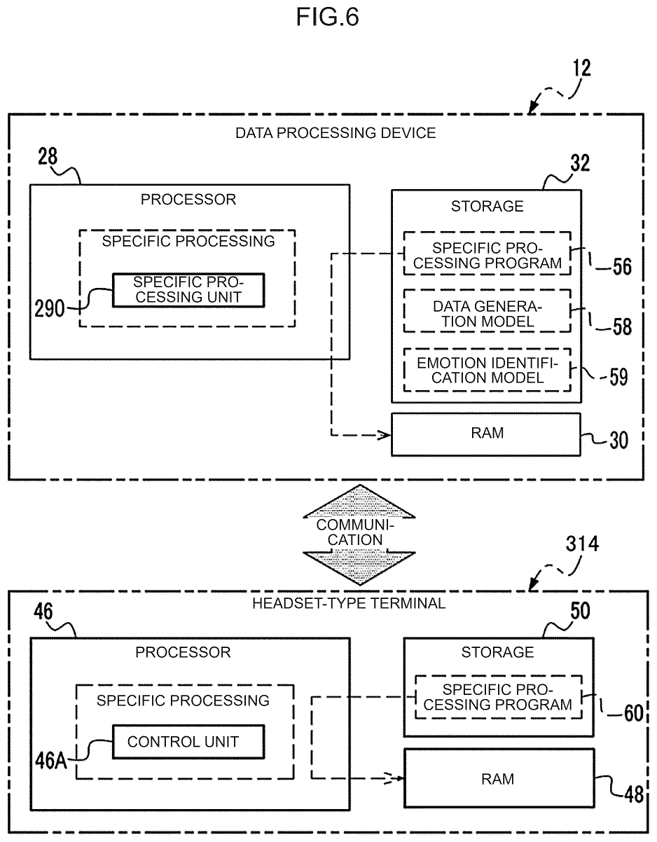

FIG. 6 shows an example of the main functions of the data processing device 12 and the headset-type terminal 314. As shown in FIG. 6, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the headset-type terminal 314, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The headset-type terminal 314 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the headset-type terminal 314. In the headset-type terminal 314, the control unit 46A causes the speaker 240 and the display 343 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 310 according to the third embodiment performs the same processing as the data processing system 10 according to the first embodiment. The processing by the data processing system 310 is executed by the specific processing unit 290 of the data processing device 12 or the control unit 46A of the headset-type terminal 314, but it may be executed by both the specific processing unit 290 of the data processing device 12 and the control unit 46A of the headset-type terminal 314. Additionally, the specific processing unit 290 of the data processing device 12 acquires or collects necessary information for processing from the headset-type terminal 314 or external devices, and the headset-type terminal 314 acquires or collects necessary information for processing from the data processing device 12 or external devices.

Each of the plurality of elements including the aforementioned imaging unit, diagnosis unit, notification unit, cooperation unit, consent unit, proposal unit, recording unit, sharing unit, and reminder unit is implemented by at least one of, for example, the headset-type terminal 314 and the data processing device 12. For example, the imaging unit captures images of the user's oral cavity using the camera 42 of the headset-type terminal 314. The diagnosis unit is implemented, for example, by a specific processing unit 290 of the data processing device 12, and AI analyzes the transmitted images and diagnoses the condition of the oral cavity. The notification unit is implemented, for example, by a control unit 46A of the headset-type terminal 314, and sends notifications to the user based on the diagnosis result. The cooperation unit is implemented, for example, by the specific processing unit 290 of the data processing device 12, and transmits the diagnosis result to the company's health management system. The consent unit is implemented, for example, by the control unit 46A of the headset-type terminal 314, and obtains the user's consent. The proposal unit is implemented, for example, by the specific processing unit 290 of the data processing device 12, and provides specific advice or treatment suggestions based on the diagnosis result. The recording unit is implemented, for example, by the specific processing unit 290 of the data processing device 12, and records the diagnosis result at regular intervals and compares it with past data. The sharing unit is implemented, for example, by the specific processing unit 290 of the data processing device 12, and shares the diagnosis result with a professional dentist. The reminder unit is implemented, for example, by the control unit 46A of the headset-type terminal 314, and sends reminders to receive appropriate treatment. The correspondence between each unit and the device or control unit is not limited to the examples described above and various modifications are possible.

### Fourth Embodiment

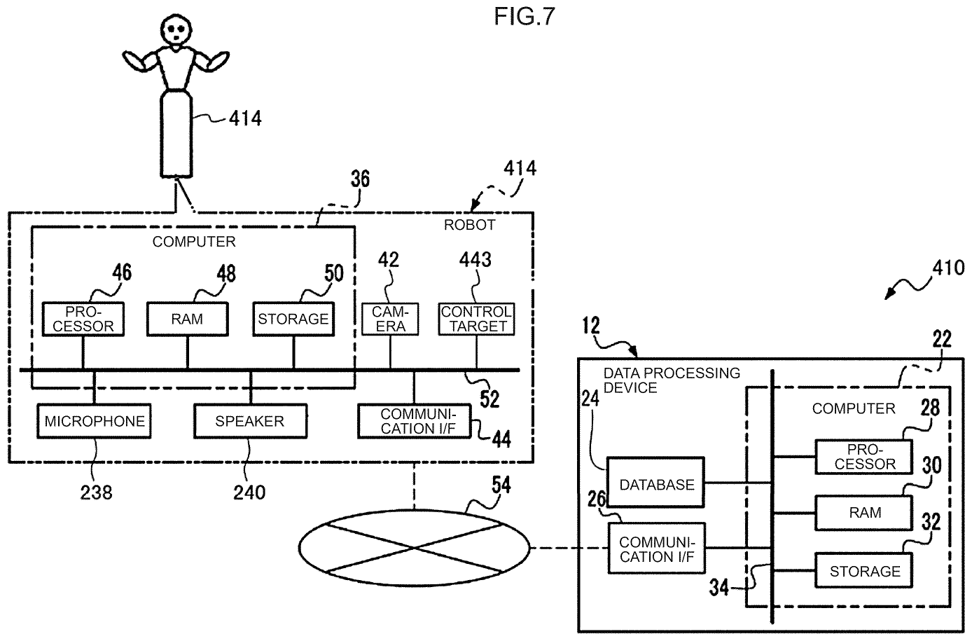

FIG. 7 shows an example configuration of a data processing system 410 according to the fourth embodiment.

As shown in FIG. 7, the data processing system 410 comprises a data processing device 12 and a robot 414. An example of the data processing device 12 is a server.

The data processing device 12 comprises a computer 22, a database 24, and a communication I/F 26. The computer 22 comprises a processor 28, RAM 30, and storage 32. The processor 28, RAM 30, and storage 32 are connected to a bus 34. Additionally, the database 24 and communication I/F 26 are also connected to the bus 34. The communication I/F 26 is connected to a network 54. Examples of the network 54 include a WAN and/or a LAN, among others.

The robot 414 comprises a computer 36, a microphone 238, a speaker 240, a camera 42, a communication I/F 44, and a control target 443. The computer 36 comprises a processor 46, RAM 48, and storage 50. The processor 46, RAM 48, and storage 50 are connected to a bus 52. The microphone 238, speaker 240, camera 42, and control target 443 are also connected to the bus 52.

The microphone 238 accepts voice from the user, accepting instructions, among others, from the user. The microphone 238 captures the voice emitted by the user, converts the captured voice into voice data, and outputs it to the processor 46. The speaker 240 outputs sound according to instructions from the processor 46.

The camera 42 is a small digital camera equipped with optical systems such as lenses, apertures, and shutters, as well as imaging elements such as CMOS image sensors or CCD image sensors, and captures the surroundings of the user (e.g., an imaging range defined by an angle of view equivalent to the typical field of view of a healthy person).

The communication I/F 44 is connected to the network 54. The communication I/F 44 and 26 manage the exchange of various information between the processor 46 and the processor 28 via the network 54. The exchange of various information between the processor 46 and the processor 28 using the communication I/F 44 and 26 is conducted securely.

The control target 443 includes a display device, LEDs for the eyes, and motors for driving arms, hands, and feet, among others. The posture and gestures of the robot 414 are controlled by controlling the motors for the arms, hands, and feet, among others. Some emotions of the robot 414 can be expressed by controlling these motors. Additionally, the expression of the robot 414 can be expressed by controlling the lighting state of the LEDs for the eyes of the robot 414.

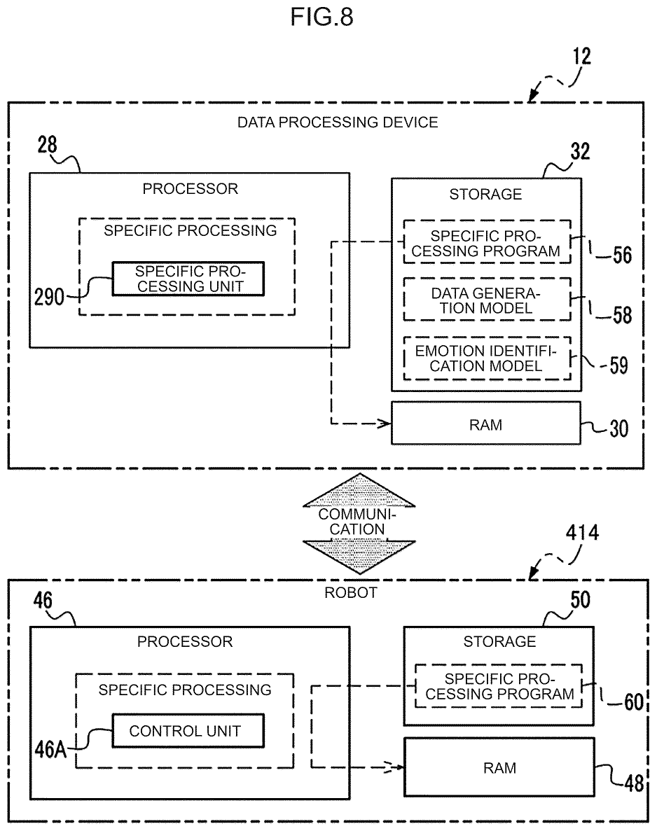

FIG. 8 shows an example of the main functions of the data processing device 12 and the robot 414. As shown in FIG. 8, specific processing is performed in the data processing device 12 by the processor 28. The storage 32 stores a specific processing program 56.

The processor 28 reads the specific processing program 56 from the storage 32 and executes it on the RAM 30. The specific processing is realized by the processor 28 operating as a specific processing unit 290 according to the specific processing program 56 executed on the RAM 30.

The storage 32 stores a data generation model 58 and an emotion identification model 59. The data generation model 58 and emotion identification model 59 are used by the specific processing unit 290. The specific processing unit 290 can estimate the user's emotions using the emotion identification model 59 and perform specific processing using the user's emotions. The emotion estimation function (emotion identification function) using the emotion identification model 59 includes estimating and predicting the user's emotions, but is not limited to such examples. Furthermore, emotion estimation and prediction may include, for example, emotion analysis.

In the robot 414, specific processing is performed by the processor 46. The storage 50 stores a specific program 60. The processor 46 reads the specific program 60 from the storage 50 and executes it on the RAM 48. The specific processing is realized by the processor 46 operating as a control unit 46A according to the specific program 60 executed on the RAM 48. The robot 414 may also have similar data generation models and emotion identification models as the data generation model 58 and emotion identification model 59, and perform the same processing as the specific processing unit 290 using these models.

Other devices besides the data processing device 12 may have the data generation model 58. For example, a server device may have the data generation model 58. In this case, the data processing device 12 communicates with the server device having the data generation model 58 to obtain processing results (e.g., prediction results) using the data generation model 58. The data processing device 12 may be a server device or a terminal device owned by the user (e.g., a mobile phone, robot, home appliance, etc.).

The specific processing unit 290 sends the results of specific processing to the robot 414. In the robot 414, the control unit 46A causes the speaker 240 and the control target 443 to output the results of specific processing. The microphone 238 acquires voice indicating user input in response to the results of specific processing. The control unit 46A sends the voice data indicating user input acquired by the microphone 238 to the data processing device 12. In the data processing device 12, the specific processing unit 290 acquires the voice data.

The data generation model 58 is a so-called generative AI. An example of the data generation model 58 is a generative AI such as ChatGPT. The data generation model 58 is obtained by performing deep learning on a neural network. The data generation model 58 receives prompts containing instructions and inference data such as voice data indicating voice, text data indicating text, and image data indicating images (e.g., still image data or video data). The data generation model 58 performs inference according to the instructions indicated by the prompt on the input inference data and outputs the inference results in one or more data formats such as voice data, text data, or image data. The data generation model 58 includes, for example, text generation AI, image generation AI, and multimodal generation AI. Here, inference refers to, for example, analysis, classification, prediction, and/or summarization. The specific processing unit 290 performs the specific processing described above using the data generation model 58. The data generation model 58 may be a fine-tuned model that outputs inference results from prompts without instructions, and in this case, the data generation model 58 can output inference results from prompts without instructions. The data processing device 12 and the like may include multiple types of data generation models 58, and the data generation model 58 may include AI other than generative AI. AI other than generative AI may include, for example, linear regression, logistic regression, decision trees, random forests, support vector machines (SVM), k-means clustering, convolutional neural networks (CNN), recurrent neural networks (RNN), generative adversarial networks (GAN), or naive Bayes, among others, and can perform various processing but are not limited to such examples. Additionally, AI may be an AI agent. Furthermore, when processing is performed by AI in each part described above, the processing may be performed partially or entirely by AI but is not limited to such examples. Additionally, processing implemented by AI including generative AI may be replaced with rule-based processing, and rule-based processing may be replaced with processing implemented by AI including generative AI.

The data processing system 410 according to the fourth embodiment performs the same processing as the data processing system 10 according to the first embodiment. The processing by the data processing system 410 is executed by the specific processing unit 290 of the data processing device 12 or the control unit 46A of the robot 414, but it may be executed by both the specific processing unit 290 of the data processing device 12 and the control unit 46A of the robot 414. Additionally, the specific processing unit 290 of the data processing device 12 acquires or collects necessary information for processing from the robot 414 or external devices, and the robot 414 acquires or collects necessary information for processing from the data processing device 12 or external devices.

Each of the plurality of elements including the aforementioned imaging unit, diagnosis unit, notification unit, cooperation unit, consent unit, proposal unit, recording unit, sharing unit, and reminder unit is implemented by at least one of, for example, the robot 414 and the data processing device 12. For example, the imaging unit captures images of the user's oral cavity using the camera 42 of the robot 414. The diagnosis unit is implemented, for example, by a specific processing unit 290 of the data processing device 12, and AI analyzes the transmitted images and diagnoses the condition of the oral cavity. The notification unit is implemented, for example, by a control unit 46A of the robot 414, and sends notifications to the user based on the diagnosis result. The cooperation unit is implemented, for example, by the specific processing unit 290 of the data processing device 12, and transmits the diagnosis result to the company's health management system. The consent unit is implemented, for example, by the control unit 46A of the robot 414, and obtains the user's consent. The proposal unit is implemented, for example, by the specific processing unit 290 of the data processing device 12, and provides specific advice or treatment suggestions based on the diagnosis result. The recording unit is implemented, for example, by the specific processing unit 290 of the data processing device 12, and records the diagnosis result at regular intervals and compares it with past data. The sharing unit is implemented, for example, by the specific processing unit 290 of the data processing device 12, and shares the diagnosis result with a professional dentist. The reminder unit is implemented, for example, by the control unit 46A of the robot 414, and sends reminders to receive appropriate treatment. The correspondence between each unit and the device or control unit is not limited to the examples described above and various modifications are possible.

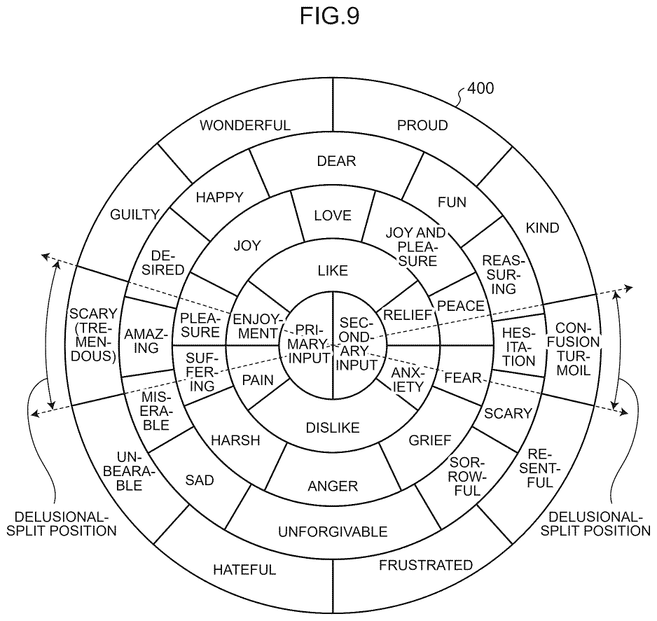

Note that the emotion identification model 59 as an emotion engine may determine the user's emotions according to a specific mapping. Specifically, the emotion identification model 59 may determine the user's emotions according to an emotion map, which is a specific mapping (see FIG. 9). Similarly, the emotion identification model 59 may determine the robot's emotions, and the specific processing unit 290 may perform specific processing using the robot's emotions.

FIG. 9 is a diagram showing an emotion map 400 where multiple emotions are mapped. In the emotion map 400, emotions are arranged concentrically radiating from the center. The closer to the center of the concentric circles, the more primitive the state of emotions is arranged. On the outer side of the concentric circles, emotions representing states and behaviors arising from mood are arranged. Emotions encompass concepts including emotional and mental states. On the left side of the concentric circles, emotions generally generated from reactions occurring in the brain are arranged. On the right side of the concentric circles, emotions generally induced by situational judgment are arranged. On the top and bottom of the concentric circles, emotions generated from reactions occurring in the brain and induced by situational judgment are arranged. Additionally, on the upper side of the concentric circles, “pleasant” emotions are arranged, and on the lower side, “unpleasant” emotions are arranged. In this way, in the emotion map 400, multiple emotions are mapped based on the structure from which emotions arise, and emotions that tend to occur simultaneously are mapped nearby.

These emotions are distributed in the 3 o'clock direction of the emotion map 400, and they usually move back and forth around reassurance and anxiety. In the right half of the emotion map 400, situational recognition takes precedence over internal sensations, giving a calm impression.

The inner side of the emotion map 400 represents the mind, and the outer side represents behavior, so the further out on the emotion map 400, the more visible (expressed in behavior) emotions become.

Here, human emotions are based on various balances like posture and blood sugar levels, and when these balances move away from the ideal, they indicate discomfort, and when they approach the ideal, they indicate comfort. In robots, cars, motorcycles, etc., emotions can be created based on various balances like posture and battery level, indicating discomfort when these balances move away from the ideal and comfort when they approach the ideal. The emotion map may be generated based on Dr. Mitsuyoshi's emotion map (Research on speech emotion recognition and brain physiological signal analysis systems related to emotions, Tokushima University, Doctoral dissertation: https://ci.nii.ac.jp/naid/500000375379). In the left half of the emotion map, emotions belonging to the domain called “reactions,” where sensations take precedence, are aligned. Additionally, in the right half of the emotion map, emotions belonging to the domain called “situations,” where situational recognition takes precedence, are aligned.

In the emotion map, two emotions that promote learning are defined. One is a negative emotion around “repentance” or “reflection” on the situation side. In other words, when a negative emotion arises in the robot, like “I never want to feel this way again” or “I don't want to be scolded again.” The other is an emotion around “desire” on the reaction side, which is positive. In other words, it is a positive feeling like “I want more” or “I want to know more.”

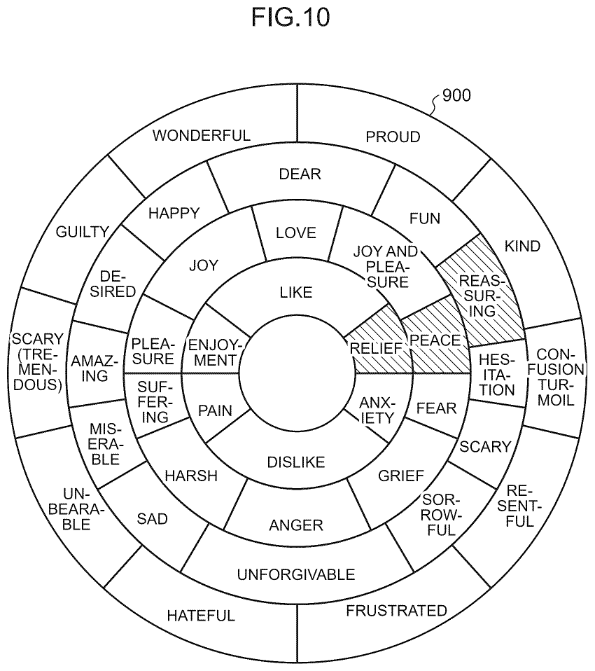

The emotion identification model 59 inputs user input into a pre-learned neural network, acquires emotion values indicating each emotion shown in the emotion map 400, and determines the user's emotions. This neural network is pre-learned based on multiple training data consisting of user input and combinations of emotion values indicating each emotion shown in the emotion map 400. Additionally, this neural network is learned so that emotions placed near each other in the emotion map 900 shown in FIG. 10 have similar values. FIG. 10 shows an example where multiple emotions like “reassured,” “calm,” and “confident” have similar emotion values.

In the above embodiments, an example form where specific processing is performed by a single computer 22 was described, but the technology disclosed herein is not limited to this, and distributed processing for specific processing by multiple computers including the computer 22 may be performed.

In the above embodiments, an example form where the specific processing program 56 is stored in the storage 32 was described, but the technology disclosed herein is not limited to this. For example, the specific processing program 56 may be stored in portable non-transitory storage media readable by a computer, such as a USB (Universal Serial Bus) memory. The specific processing program 56 stored in non-transitory storage media is installed in the computer 22 of the data processing device 12. The processor 28 executes specific processing according to the specific processing program 56.

Additionally, the specific processing program 56 may be stored in a storage device, such as a server connected to the data processing device 12 via the network 54, and downloaded and installed on the computer 22 in response to requests from the data processing device 12.

Furthermore, it is not necessary to store all of the specific processing program 56 in storage devices such as servers connected to the data processing device 12 via the network 54 or all in the storage 32, and a part of the specific processing program 56 may be stored.

Various processors, as shown next, can be used as hardware resources for executing specific processing. As processors, general-purpose processors that function as hardware resources for executing specific processing by executing software, i.e., programs, such as a CPU, can be mentioned. Additionally, as processors, dedicated electrical circuits with circuit configurations specially designed to execute specific processing, such as FPGA (Field-Programmable Gate Array), PLD (Programmable Logic Device), or ASIC (Application Specific Integrated Circuit), can be mentioned. Each processor has a built-in or connected memory, and each processor executes specific processing using the memory.

Hardware resources for executing specific processing may be composed of one of these various processors or a combination of two or more processors of the same or different types (e.g., a combination of multiple FPGAs or a combination of a CPU and FPGA). Additionally, hardware resources for executing specific processing may be a single processor.

As an example of composing with a single processor, firstly, there is a form where one or more CPUs and software are combined to constitute a single processor, which functions as hardware resources for executing specific processing. Secondly, there is a form using a processor, such as SoC (System-on-a-chip), that realizes the function of an entire system including multiple hardware resources for executing specific processing with a single IC chip. In this way, specific processing is realized using one or more of the various processors as hardware resources.

Furthermore, as a hardware structure of these various processors, more specifically, electrical circuits combined with circuit elements such as semiconductor elements can be used. Additionally, the specific processing described above is merely one example. Therefore, it goes without saying that unnecessary steps may be deleted, new steps may be added, or the order of processing may be changed within the scope not departing from the gist.

Additionally, in the examples described above, the explanation was divided into the first embodiment to the fourth embodiment, but parts or all of these embodiments may be combined. Additionally, the smart device 14, smart glasses 214, headset-type terminal 314, and robot 414 are examples, and each may be combined, or other devices may be used.

The descriptions and drawings shown above are detailed explanations of parts related to the technology disclosed herein and are merely examples of the technology disclosed herein. For example, the explanations regarding configurations, functions, actions, and effects above are explanations regarding examples of configurations, functions, actions, and effects of parts related to the technology disclosed herein. Therefore, it goes without saying that within the scope not departing from the gist of the technology disclosed herein, unnecessary parts may be deleted, new elements may be added, or replacements may be made to the descriptions and drawings shown above. Additionally, to avoid complexity and facilitate understanding of parts related to the technology disclosed herein, explanations concerning technical common knowledge and the like that do not require special explanation for enabling the implementation of the technology disclosed herein are omitted in the descriptions and drawings shown above.

All documents, patent applications, and technical standards described in this specification are incorporated by reference to the same extent as if each document, patent application, and technical standard were specifically and individually stated to be incorporated by reference in this specification.
(Supplementary Note 1)A system comprising: an imaging unit configured to capture images of the oral cavity of a user using a PC's internal camera; a diagnosis unit configured to diagnose the images captured by the imaging unit using AI; and a notification unit configured to send the user the results of the oral check or a prompt to visit a medical institution based on the diagnosis result obtained by the diagnosis unit.
(Supplementary Note 2)The system according to Supplementary Note 1, further comprising a cooperation unit configured to cooperate with a company regarding the diagnosis result.
(Supplementary Note 3)The system according to Supplementary Note 1, further comprising a consent unit configured to obtain the user's consent.
(Supplementary Note 4)The system according to Supplementary Note 1, further comprising a proposal unit configured to provide specific advice or treatment suggestions based on the diagnosis result.
(Supplementary Note 5)The system according to Supplementary Note 1, further comprising a recording unit configured to record the diagnosis result at regular intervals and compare it with past data.
(Supplementary Note 6)The system according to Supplementary Note 1, further comprising a sharing unit configured to share the diagnosis result with a professional dentist and obtain expert opinions.
(Supplementary Note 7)The system according to Supplementary Note 1, further comprising a reminder unit configured to send reminders to receive appropriate treatment.
(Supplementary Note 8)The system according to Supplementary Note 1, wherein the imaging unit is configured to estimate the user's emotion and adjust the timing of imaging based on the estimated emotional state of the user.
(Supplementary Note 9)The system according to Supplementary Note 1, wherein the imaging unit is configured to add a function to emphasize and capture specific areas of the user's oral cavity during imaging.
(Supplementary Note 10)The system according to Supplementary Note 1, wherein the imaging unit is configured to measure the humidity and temperature of the user's oral cavity during imaging and utilize the measurements for diagnosis.
(Supplementary Note 11)The system according to Supplementary Note 1, wherein the imaging unit is configured to estimate the user's emotion and adjust the frequency of imaging based on the estimated emotional state of the user.
(Supplementary Note 12)The system according to Supplementary Note 1, wherein the imaging unit is configured to capture the user's entire face during imaging and comprehensively determine the user's health condition based on complexion and facial expressions.
(Supplementary Note 13)The system according to Supplementary Note 1, wherein the imaging unit is configured to record the user's voice during imaging and associate it with the health condition of the oral cavity.
(Supplementary Note 14)The system according to Supplementary Note 1, wherein the diagnosis unit is configured to estimate the user's emotion and adjust the manner of expressing the diagnosis result based on the estimated emotional state of the user.
(Supplementary Note 15)The system according to Supplementary Note 1, wherein the diagnosis unit is configured to add a function to identify the types and quantities of bacteria in the oral cavity during diagnosis.
(Supplementary Note 16)The system according to Supplementary Note 1, wherein the diagnosis unit is configured to refer to the user's past diagnosis data during diagnosis to improve diagnostic accuracy.
(Supplementary Note 17)The system according to Supplementary Note 1, wherein the diagnosis unit is configured to estimate the user's emotion and adjust the level of detail of the diagnosis result based on the estimated emotional state of the user.
(Supplementary Note 18)The system according to Supplementary Note 1, wherein the diagnosis unit is configured to correct the diagnosis result during diagnosis by considering the user's dietary habits and lifestyle.
(Supplementary Note 19)The system according to Supplementary Note 1, wherein the diagnosis unit is configured to correct the diagnosis result during diagnosis by considering the user's genetic information.
(Supplementary Note 20)The system according to Supplementary Note 1, wherein the notification unit is configured to estimate the user's emotion and adjust the timing of notification based on the estimated emotional state of the user.
(Supplementary Note 21)The system according to Supplementary Note 1, wherein the notification unit is configured to select a notification method according to the user's preferences at the time of notification.
(Supplementary Note 22)The system according to Supplementary Note 1, wherein the notification unit is configured to select the optimal notification timing by considering the user's schedule at the time of notification.
(Supplementary Note 23)The system according to Supplementary Note 1, wherein the notification unit is configured to estimate the user's emotion and adjust the content of notification based on the estimated emotional state of the user.
(Supplementary Note 24)The system according to Supplementary Note 1, wherein the notification unit is configured to determine the priority of notification according to the user's health condition at the time of notification.
(Supplementary Note 25)The system according to Supplementary Note 1, wherein the notification unit is configured to add a function to send notifications to the user's family or medical personnel at the time of notification.
(Supplementary Note 26)The system according to Supplementary Note 2, wherein the cooperation unit is configured to estimate the user's emotion and adjust the content of cooperation with the company based on the estimated emotional state of the user.
(Supplementary Note 27)The system according to Supplementary Note 2, wherein the cooperation unit is configured to integrate with the company's health management system and share data at the time of cooperation.
(Supplementary Note 28)The system according to Supplementary Note 2, wherein the cooperation unit is configured to cooperate with the company's welfare program to support health management at the time of cooperation.
(Supplementary Note 29)The system according to Supplementary Note 2, wherein the cooperation unit is configured to estimate the user's emotion and adjust the frequency of cooperation based on the estimated emotional state of the user.
(Supplementary Note 30)The system according to Supplementary Note 2, wherein the cooperation unit is configured to introduce encryption technology to protect the user's privacy at the time of cooperation.
(Supplementary Note 31)The system according to Supplementary Note 2, wherein the cooperation unit is configured to anonymize the user's health data and provide it to the company at the time of cooperation.
(Supplementary Note 32)The system according to Supplementary Note 3, wherein the consent unit is configured to estimate the user's emotion and adjust the method of obtaining consent based on the estimated emotional state of the user.
(Supplementary Note 33)The system according to Supplementary Note 3, wherein the consent unit is configured to provide detailed explanations to the user at the time of obtaining consent to deepen understanding.
(Supplementary Note 34)The system according to Supplementary Note 3, wherein the consent unit is configured to record the user's consent history at the time of obtaining consent so that it can be referenced later.
(Supplementary Note 35)The system according to Supplementary Note 3, wherein the consent unit is configured to estimate the user's emotion and adjust the timing of obtaining consent based on the estimated emotional state of the user.
(Supplementary Note 36)The system according to Supplementary Note 3, wherein the consent unit is configured to provide explanations according to the user's language and culture at the time of obtaining consent.
(Supplementary Note 37)The system according to Supplementary Note 3, wherein the consent unit is configured to request consent from the user's family or medical personnel at the time of obtaining consent.
(Supplementary Note 38)The system according to Supplementary Note 4, wherein the proposal unit is configured to estimate the user's emotion and adjust the content of suggestions based on the estimated emotional state of the user.
(Supplementary Note 39)The system according to Supplementary Note 4, wherein the proposal unit is configured to refer to the user's past diagnosis data at the time of suggestion to propose the optimal treatment method.
(Supplementary Note 40)The system according to Supplementary Note 4, wherein the proposal unit is configured to provide specific advice based on the user's lifestyle and dietary habits at the time of suggestion.
(Supplementary Note 41)The system according to Supplementary Note 4, wherein the proposal unit is configured to estimate the user's emotion and determine the priority of suggestions based on the estimated emotional state of the user.
(Supplementary Note 42)The system according to Supplementary Note 4, wherein the proposal unit is configured to customize the content of suggestions according to the user's health condition at the time of suggestion.
(Supplementary Note 43)The system according to Supplementary Note 4, wherein the proposal unit is configured to share the content of suggestions with the user's family or medical personnel at the time of suggestion.
(Supplementary Note 44)The system according to Supplementary Note 5, wherein the recording unit is configured to estimate the user's emotion and adjust the content of records based on the estimated emotional state of the user.
(Supplementary Note 45)The system according to Supplementary Note 5, wherein the recording unit is configured to analyze changes in health condition by comparing the user's past diagnosis data at the time of recording.
(Supplementary Note 46)The system according to Supplementary Note 5, wherein the recording unit is configured to record the user's lifestyle and dietary data together at the time of recording.
(Supplementary Note 47)The system according to Supplementary Note 5, wherein the recording unit is configured to estimate the user's emotion and adjust the frequency of recording based on the estimated emotional state of the user.
(Supplementary Note 48)The system according to Supplementary Note 5, wherein the recording unit is configured to store the user's health data in the cloud at the time of recording so that it can be accessed at any time.
(Supplementary Note 49)The system according to Supplementary Note 5, wherein the recording unit is configured to anonymize the user's health data and provide it to research institutions at the time of recording.
(Supplementary Note 50)The system according to Supplementary Note 6, wherein the sharing unit is configured to estimate the user's emotion and adjust the content of sharing based on the estimated emotional state of the user.
(Supplementary Note 51)The system according to Supplementary Note 6, wherein the sharing unit is configured to provide detailed diagnosis data to a professional dentist at the time of sharing.
(Supplementary Note 52)The system according to Supplementary Note 6, wherein the sharing unit is configured to provide the user's past diagnosis data together at the time of sharing to obtain comprehensive opinions.
(Supplementary Note 53)The system according to Supplementary Note 6, wherein the sharing unit is configured to estimate the user's emotion and adjust the timing of sharing based on the estimated emotional state of the user.
(Supplementary Note 54)The system according to Supplementary Note 6, wherein the sharing unit is configured to introduce encryption technology to protect the user's privacy at the time of sharing.
(Supplementary Note 55)The system according to Supplementary Note 6, wherein the sharing unit is configured to anonymize the user's health data and provide it to a professional dentist at the time of sharing.
(Supplementary Note 56)The system according to Supplementary Note 7, wherein the reminder unit is configured to estimate the user's emotion and adjust the content of reminders based on the estimated emotional state of the user.
(Supplementary Note 57)The system according to Supplementary Note 7, wherein the reminder unit is configured to select the optimal timing for sending reminders by considering the user's schedule at the time of sending reminders.
(Supplementary Note 58)The system according to Supplementary Note 7, wherein the reminder unit is configured to select a sending method according to the user's preferences at the time of sending reminders.
(Supplementary Note 59)The system according to Supplementary Note 7, wherein the reminder unit is configured to estimate the user's emotion and adjust the frequency of reminders based on the estimated emotional state of the user.
(Supplementary Note 60)The system according to Supplementary Note 7, wherein the reminder unit is configured to customize the content of reminders according to the user's health condition at the time of sending reminders.
(Supplementary Note 61)The system according to Supplementary Note 7, wherein the reminder unit is configured to add a function to send reminders to the user's family or medical personnel at the time of sending reminders.

What is claimed is:

1. A system comprising:
a communication interface configured to communicate with a client terminal via a packet-switched network;
a memory storing a feature extraction model obtained by deep learning on a convolutional neural network, and an emotion identification model; and
circuitry configured to:
receive, from the client terminal via the communication interface, image data captured by a CMOS image sensor of the client terminal;
extract a feature vector from the image data by inputting the image data into the feature extraction model, the feature extraction model comprising a convolutional neural network configured to output a multidimensional tensor representing image features;
classify the feature vector using a classification layer to generate a classification label and a confidence score;
estimate an emotion of a user by applying the emotion identification model to sensor data received from the client terminal via the communication interface; and
transmit, to the client terminal via the communication interface and the packet-switched network, output data generated based on the classification label and the estimated emotion, the output data causing the client terminal to present the output data to the user.

2. The system according to claim 1, wherein the image data comprises oral cavity image data of the user, and wherein the classification label comprises at least one of a cavity detection label, a periodontal disease detection label, or an oral inflammation score.

3. The system according to claim 1, wherein the circuitry is further configured to perform preprocessing on the image data before inputting the image data into the feature extraction model, the preprocessing comprising at least one of brightness correction, contrast correction, noise removal, or region extraction.

4. The system according to claim 1, wherein the feature extraction model further comprises a Transformer-based multimodal model configured to process the image data using a self-attention mechanism.

5. The system according to claim 1, wherein the circuitry is further configured to segment the image data into a plurality of regions by applying a segmentation model comprising at least one of a U-Net or a Mask R-CNN, and to generate the classification label for each of the plurality of regions.

6. The system according to claim 1, wherein the circuitry is further configured to determine an action recommendation based on the confidence score by applying at least one of threshold judgment or rule-based branching.

7. The system according to claim 1, wherein the emotion identification model is configured to receive at least one of facial expression image data, voice data, or biometric sensor data comprising heart rate or skin conductance values, and to output an emotion label and an emotion score.

8. The system according to claim 1, wherein the circuitry is further configured to adjust a timing of receiving the image data from the client terminal based on the estimated emotion, such that when the estimated emotion indicates stress, the circuitry delays a request for the image data, and when the estimated emotion indicates relaxation, the circuitry flexibly adjusts the timing.

9. The system according to claim 1, wherein the circuitry is further configured to adjust a frequency of receiving the image data from the client terminal based on the estimated emotion, such that when the estimated emotion indicates stress, the circuitry reduces the frequency, and when the estimated emotion indicates relaxation, the circuitry increases the frequency.

10. The system according to claim 1, wherein the circuitry is further configured to adjust a level of detail of the output data based on the estimated emotion, such that when the estimated emotion indicates stress, the circuitry generates the output data in a simplified format, and when the estimated emotion indicates relaxation, the circuitry generates the output data in a detailed format.

11. The system according to claim 1, wherein the circuitry is further configured to transmit the output data to an external system via the communication interface using an encrypted communication channel, the external system comprising a health management server.

12. The system according to claim 1, wherein the circuitry is further configured to obtain consent data from the user via the client terminal before transmitting the output data to an external system, and to store a consent history in association with the output data.

13. The system according to claim 1, wherein the circuitry is further configured to generate recommendation data comprising at least one of a treatment suggestion or a self-care advice based on the classification label and the confidence score.

14. The system according to claim 1, wherein the circuitry is further configured to store the classification label and the confidence score in a database in association with a timestamp, and to compare a current classification result with past classification results stored in the database to detect a trend.

15. The system according to claim 1, wherein the circuitry is further configured to transmit the output data to a terminal of a specialist via the communication interface, and to receive feedback data from the specialist.

16. The system according to claim 1, wherein the circuitry is further configured to select a notification method for transmitting the output data based on a user preference, the notification method comprising at least one of email, SMS, or application notification.

17. The system according to claim 1, wherein the circuitry is further configured to determine a priority of the output data based on the confidence score, such that when the confidence score exceeds a threshold, the circuitry assigns a high priority to the output data.

18. A system comprising:
a communication interface configured to communicate, via a packet-switched network conforming to at least one of a 5G, Wi-Fi, or Bluetooth communication standard, with a client terminal comprising a touch panel, a microphone, a speaker, a CMOS image sensor, and a display;
a processor;
a random-access memory;
a memory storing a feature extraction model obtained by deep learning on a convolutional neural network, and an emotion identification model;
a database; and
circuitry configured to:
receive, from the client terminal via the communication interface, image data captured by the CMOS image sensor;
extract a feature vector from the image data by inputting the image data into the feature extraction model, the feature extraction model comprising a convolutional neural network configured to output a multidimensional tensor;
classify the feature vector using a classification layer to generate a classification label and a confidence score, and store the classification label and the confidence score in the database;
estimate an emotion of a user by applying the emotion identification model to at least one of voice data captured by the microphone or image data captured by the CMOS image sensor;
adjust at least one of a format, a level of detail, or a priority of output data based on the estimated emotion; and
transmit the output data to the client terminal via the communication interface, the output data causing the client terminal to present the output data to the user via at least one of the display or the speaker.

19. The system according to claim 18, wherein the image data comprises oral cavity image data of the user, and wherein the classification label comprises at least one of an abnormality detection label indicating presence of a cavity, a periodontal disease indicator, or an inflammation score.

20. A method performed by circuitry of a data processing system comprising a communication interface and a memory storing a feature extraction model obtained by deep learning on a convolutional neural network and an emotion identification model, the method comprising:
receiving, from a client terminal via the communication interface and a packet-switched network, image data captured by a CMOS image sensor of the client terminal;
extracting a feature vector from the image data by inputting the image data into the feature extraction model, the feature extraction model comprising a convolutional neural network configured to output a multidimensional tensor representing image features;
classifying the feature vector using a classification layer to generate a classification label and a confidence score;
estimating an emotion of a user by applying the emotion identification model to sensor data received from the client terminal via the communication interface; and
transmitting, to the client terminal via the communication interface and the packet-switched network, output data generated based on the classification label and the estimated emotion, the output data causing the client terminal to present the output data to the user.
